(19) United States
(12) Patent Application Publication
(10) Pub. No.: US 2023/0149516 A1
(43) Pub. Date: May 18, 2023

(54) USE OF PEPTIDYLGLYCINE ALPHA-AMIDATING MONOOXYGENASE (PAM) FOR THERAPEUTIC PURPOSE

(71) Applicant: PAM THERAGNOSITICS GMBH, Hennigsdorf (DE)

(72) Inventor: Andreas BERGMANN, Berlin (DE)

(73) Assignee: PAM THERAGNOSTICS GMBH, Hennigsdorf (DE)

(21) Appl. No.: 17/802,338

(22) PCT Filed: Feb. 26, 2021
(86) PCT No.: PCT/EP2021/054869

§ 371 Date: Aug. 25, 2022

(30) Foreign Application Priority Data
Feb. 26, 2020 (WO) 20159647.5

(51) Int. Cl. A61K 38/43; A61P 13/12; A61P 31/00; A61P 9/00

(52) U.S. Cl. CPC A61K 38/43; A61P 13/12; A61P 31/00; A61P 9/00

(57) ABSTRACT

The present invention is directed to peptidylglycine alpha-amidating monooxygenase (PAM) for use as a medicament and the treatment of a subject, wherein said treatment comprises: reducing the potential or risk for a disease or disorder, and/or reducing the occurrence of a disease or disorder, and/or reducing the severity of a disease or disorder.

The present invention is directed to the use of peptidylglycine alpha-amidating monooxygenase (PAM) as medicament.

### STATE OF THE ART

Biologically active peptide hormones fulfill the function as signaling molecules. Most bioactive peptide hormones are synthesized from larger, inactive precursor peptides. During their biosynthesis, those peptides undergo several co- and posttranslational modifications, including cleavage of signal peptides, endoproteolytic cleavage of the precursor pro-peptides by specific endopeptidases mostly at pairs of basic residues, removal of basic residues by carboxypeptidases, formations of disulfide bonds and N- and O-glycosylation (Eipper et al. 1993. Protein Science 2(4): 489-97). More than half of the known neural and endocrine peptides require an additional modification step to gain full biological activity involving the formation of a c-terminal alpha-amide group (Guembe, et al. 1999. J Histochem Cytochem 47(5): 623-36). This final step of peptide hormone biosynthesis involves the action of the bifunctional enzyme peptidylglycine alpha-amidating monooxygenase (PAM). PAM specifically recognizes c-terminal glycine residues in its substrates, cleaves glyoxylate from the peptide's c-terminal glycine residue in a two-step enzymatic reaction leading to the formation of c-terminally alpha-amidated peptide hormones, wherein the resulting alpha-amide group originates from the cleaved c-terminal glycine (Prigge et al. 2004. Science 304(5672): 864-67). This amidation reaction takes place in the lumen of secretory granules prior to exocytosis of the amidated product (Martinez and Treston 1996. Molecular and Cellular Endocrinol 123: 113-17). Alpha-amidated peptides are for example adrenomedullin, substance P, vasopressin, neuropeptide Y, Amylin, calcitonin, neurokinin A and others. However, previously it was demonstrated that PAM can also catalyze the formation of alpha-amides from glycinated substrates of non-peptide character, e.g. N-fatty acyl-glycines, which are converted by PAM to primary fatty acid amides (PFAMs) like oleamide. The identified and purified peptidyl-glycine amidating activities were shown to be dependent on copper and ascorbate (Emeson et al. 1984. Journal of Neuroscience: 2604-13; Kumar et al. 2016 J Mol Endocrinol 56(4):T63-76; Wand et al. 1985. Neuroendocrinology 41: 482-89).

In humans, the PAM gene is located at chromosome 5q21.1 having a length of 160 kb containing 25 known exons (Gaier et al. 2014. BMC Endocrine Disorders 14). At least 6 isoforms are known to be generated by alternative splicing (SEQ ID 1-6). The PAM enzyme was found to be expressed at different levels in almost all mammalian cell types, with significant expression in airway epithelium, endothelial cells, ependymal cells in the brain, adult atrium, brain, kidney, pituitary, gastrointestinal tract and reproductive tissues (Chen et al. 2018. Diabetes Obes Metab 20 Suppl 2:64-76; Oldham et al. 1992. Biochem Biophys Res Commun 184(1): 323-29; Schafer et al. 1992. J Neurosci 12(1): 222-34).

However, the highest human PAM activity was described in the pituitary, the stalk and hypothalamus. The plasma amidating activity of healthy children below 15 years was significantly higher than that of healthy adults (Wand et al. 1985 Metabolism 34(1): 1044-52).

The precursor protein (1-973 amino acids) of the largest known PAM Isoform 1 (SEQ ID No. 1) encoded by the PAM cDNA is depicted in FIG. 1. The N-terminal signal sequence (amino acids 1-20) assures direction of the nascent PAM polypeptide into the secretory lumen of endoplasmic reticulum and is subsequently cleaved co-translationally. Afterwards the PAM-pro-peptide is processed by the same machinery used for the biosynthesis of integral membrane proteins and secreted proteins including cleavage of the pro-region (amino acids 21-30), assuring proper folding, disulfide bond formation, phosphorylation and glycosylation (Bousquet-Moore et al. 2010. J Neurosci Res 88(12):2535-45).

As depicted in FIG. 1, the PAM cDNA further encodes two distinct enzymatic activities. The first enzymatic activity is named peptidyl-glycine alpha-hydroxylating monooxygenase (PHM; EC 1.14.17.3), is an enzyme, capable of catalyzing the conversion of a C-terminal glycine residue to an alpha-hydroxy-glycine. The second activity is named peptidyl-a-hydroxy-glycine alpha-amidating lyase (PAL; EC 4.3.2.5) is an enzyme capable of catalyzing the conversion of an alpha-hydroxy-glycine to an alpha-amide with subsequent glyoxylate release. The sequential action of these separate enzymatic activities results in the overall peptidyl-glycine alpha amidating activity. The first enzymatic activity (PHM) is located directly upstream of the pro-region (within of amino acids 31-494 of isoform 1 (SEQ ID No. 7)). The second catalytic activity (PAL) is located after exon 16 in isoform 1 within of amino acids 495-817 (SEQ ID No. 8).

As depicted in FIG. 2, both activities may be encoded together within of one polypeptide as a membrane-bound protein (isoforms 1, 2, 5, 6; corresponding to SEQ ID No. 1, 2, 5 and 6) as well within of one polypeptide as a soluble protein lacking the transmembrane domain (isoforms 3 and 4; corresponding to SEQ ID No. 3 and 4). While isoforms 1, 2, 5 and 6 remain in the outer plasma membrane after fusion of secretory vesicles with the plasma membrane with subsequent endocytosis and recycling or degradation, soluble PAM isoforms lacking the TMD (isoforms 3 and 4) (amino acids 864-887) are co-secreted with the peptide-hormones (Wand et al. 1985 Metabolism 34(11): 1044-52). Furthermore, prohormone convertases may convert membrane bound PAM protein into soluble PAM protein by cleavage within the flexible region (exons 25/26) connecting PAL with the TMD during the secretory pathway (Bousquet-Moore et al. 2010. J Neurosci Res 88(12):2535-45). The PHM subunit may be cleaved from soluble or membrane bound PAM within the secretory pathway by prohormone convertases that address a double-basic cleavage-site in the exon 16 region. Furthermore, during endocytosis the full-length PAM protein may be also converted into a soluble form due to the action of alpha- and gamma secretases (Bousquet-Moore et al. 2010. J Neurosci Res 88(12):2535-45). Membrane bound PAM from late endosome can be further secreted in form of exosomal vesicles.

PHM and PAL activities, as well as the activity of the full-length PAM were determined in several human tissues and body fluids. However, the separated PHM and PAL activities in soluble forms will also lead to formation of c-terminally alpha amidated products from c-terminally glycinated substrates when allowed to perform their separate reactions in the same compartment, body-fluid or in vitro experimental setup. How the transfer of the PHM hydroxylated product to the PAL takes place is not exactly understood to date. There is evidence that the hydroxylated product is released into solution and is not directly transferred from PHM to PAL (Yin et al. 2011. PLoS One 6(12):e28679). Also not clear to date is the source of PAM in circulation.

The partial reaction of PHM is depicted in FIG. 2. PHM is a copper dependent monooxygenase responsible for stereo-specific hydroxylation of the c-terminal glycine at the alpha-carbon atom. During the hydroxylation reaction ascorbate is believed to be the naturally occurring reducing agent, while the oxygen in the newly formed hydroxyl group was shown to originate from molecular oxygen. The partial reaction of the PAL is depicted in FIG. 2. The catalytic action of PAL involves proton abstraction form the PHM-formed hydroxy-glycine by a protein-backbone derived base and a nucleophilic attack of hydroxyl-group oxygen to the divalent metal leading to a cleavage of glyoxylate and formation of a c-terminal amide.

Thus the term “amidating activity”, “alpha-amidating activity”, “peptidyl-glycine alpha-amidating activity” or “PAM activity” refers to the sequential enzymatic activities of PHM and PAL, independent of the present splice variant or mixtures of splice variants or post-translationally modified PAM enzymes or soluble, separated PHM or PAL activities or soluble PHM and membrane bound PAL or combinations of all mentioned forms leading to the formation of alpha amidated products of peptide or non-peptide character from glycinated substrates of peptide or non-peptide character. In other words, the term “amidating activity”, “alpha-amidating activity”, “peptidyl-glycine alpha-amidating activity” or “PAM activity” may be described as the sequential action of enzymatic activities located within amino acids 31 to 817 in the propeptide encoded by the human PAM cDNA, independent of present splice-variants or mixtures thereof.

PAM activity was analyzed in several human tissues and body fluids of healthy specimen or those suffering from several diseases. To summarize efforts that has been done in past:

Detection of PAM activities in human body-fluids mainly involves usage of radiolabeled synthetic tripeptides such as ¹²⁵I-D-TyrValGly, ¹²⁵I-N-acetyl-TyrValGly or comparably modified tripeptides and quantification of the amidated product due to gamma-scintillation (Kapuscinski et al. 1993. Clinical Endocrinology 39(1): 51-58; Wand et al. 1985 Metabolism 34(11): 1044-52; Tsukamoto et al. 1995. Internal Medicine 34(4): 229-32. Wand et al. 1987 Neurology 37: 1057-61. Wand et al. 1985 Neuroendocrinol 41: 482-89). Furthermore, Substance P-Gly or a truncated version Neuropeptide Y-Gly were utilized as substrates for PAM activity assays (Gether et al. 1991 Mol Cell Endocrinol 79 (1-3): 53-63; Hyyppä et al. 1990 Pain 43: 163-68; Jeng et al. 1990 Analytical Biochemistry 185(2): 213-19).

The presence of alpha-amidating activity in human circulation was initially proved by Wand et al. (Wand et al. 1985 Metabolism 34(11): 1044-52). They reported no sex differences but some variations of PAM activity in certain disease states: Plasma PAM activities were increased in hypothyroid adults as well as in patients with medullary thyroid carcinoma. The activity of PAM in tissues of medullary thyroid carcinoma, pheochromocytoma and pancreatic islet tumors were shown to be elevated suggesting increased formation of amidated peptides in endocrine tumor tissues (Gether et al. 1991 Mol Cell Endocrinol 79 (1-3): 53-63; Wand et al. 1985 Neuroendocrinol 41: 482-89).

Patients suffering from multiple endocrine neoplasia type 1 (MEN-1) and pernicious anemia showed a decreased plasma PAM activity in comparison to healthy control subjects (Kapuscinski et al. 1993. Clin Endocrinol 39(1): 51-58).

The presence of amidating activity in human cerebrospinal fluid (CSF) was shown by Wand and colleagues (Wand et al. 1985 Neuroendocrinol 41: 482-89). In patients suffering from Alzheimer's disease (AD) plasma PAM activities were shown to be unaltered when compared to healthy controls, while CSF PAM activities were significantly decreased in comparison to activities from normal specimen (Wand et al. 1987 Neurology 37: 1057-61). In addition, in WO2015/103594 the presence of PAM-Protein in CSF detected by mass spectrometry of AD-patients was proposed to be reduced compared to healthy controls. Moreover, ADM-NH₂, one of the amidated products of PAM, was shown to be reduced in patients with prevalent and incident Alzheimer's disease (WO2019/154900). However, no direct association of circulating PAM activities were reported to date being associated with prediction, diagnosis or progression of AD.

Amidating activity in CSF of patients with low back pain was analyzed using 1-12 Substance P-Gly (SP-Gly) as substrate (Hyyppä et al. 1990 Pain 43: 163-68). PAM activities of patients suffering from multiple sclerosis (MS) were shown to be increased in CSF, with a significant decrease in serum (Tsukamoto et al. 1995. Internal Medicine 34(4): 229-32; WO2010/005387). An association between plasma activity of PAM and type-2-diabetes was described in (WO2014/118634).

Even though some findings were made regarding PAM activity in human body fluids and diseases or disease progression, there is no information on PAM concentrations in human body fluids, particularly in the circulation, measured with an immunoassay. As shown in the Examples, detection methods for the determination of the level of PAM (as the total amount or the activity of PAM) in a bodily fluid of a subject were established. With these assays it was shown that the level of PAM is decreased in a number of diseases as well as in patients who will develop a disease. Moreover, it was the surprising finding of the present application that the in vivo administration of recombinant PAM enzyme can be used to increase the PAM-level in the circulation, which results in an enhanced conversion of the PAM substrate adrenomedullin-glycine to mature adrenomedullin-amid. In conclusion, PAM may be used as therapy in a subject.

### DETAILED DESCRIPTION OF THE INVENTION

Subject-matter of the present application is peptidylglycine alpha-amidating monooxygenase (PAM) for use as a medicament.

Further subject-matter of the present application is PAM for use as a medicament for treatment of a subject, wherein said treatment comprises:
(i) reducing the potential or risk for a disease or disorder, and/or
(ii) reducing the occurrence of a disease or disorder, and/or
(iii) reducing the severity of a disease or disorder.

One embodiment of the present application relates to PAM for use as a medicament for treatment of a subject, wherein said disease or disorder is selected from the group comprising dementia, cardiovascular disorders, kidney diseases, cancer, inflammatory or infectious diseases and/or metabolic diseases.

Another embodiment of the present application relates to PAM for use as a medicament for treatment of a subject, wherein said subject is characterized by
a level of PAM and/or its isoforms and/or fragments thereof below a threshold and/or
a peptide-Gly/peptide-amide ratio above a threshold

in a sample of bodily fluid of said subject.

Another specific embodiment of the present application relates to PAM for use as a medicament for treatment of a subject, wherein said peptide is selected from the group of comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin, vasopressin.

Another embodiment of the present application relates to PAM for use as a medicament for treatment of a subject, wherein said subject is characterized by
an ADM-Gly/bio-ADM ratio above a threshold and/or
a bio-ADM concentration below a threshold

in a bodily fluid of said patient.

Another preferred embodiment of the present application relates to PAM for use as a medicament for treatment of a subject, wherein the level of PAM and/or its isoforms and/or fragments thereof is the total concentration of PAM and/or its isoforms and/or fragments thereof having at least 12 amino acids or the activity of PAM and/or its isoforms and/or fragments thereof.

One embodiment of the present application relates to PAM for use as a medicament for treatment of a subject, wherein the total concentration of PAM and/or its isoforms and/or fragments thereof having at least 12 amino acids or the activity of PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

Another embodiment of the present application relates to PAM for use as a medicament for treatment of a subject, wherein the sample of bodily fluid of said subject is selected from the group of blood, serum, plasma, urine, cerebrospinal fluid (CSF), and saliva.

Another specific embodiment of the present application relates to PAM for use as a medicament, wherein said PAM is selected from the group comprising isolated and/or recombinant and/or chimeric PAM.

One embodiment of the present application relates to PAM for use as a medicament, wherein said recombinant PAM is selected from the sequences comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10. One embodiment of the present application relates to PAM for use as a medicament in a subject having a level of PAM and/or its isoforms and/or fragments thereof below a threshold and/or a peptide-Gly/peptide-amide ratio above a threshold in a sample of bodily fluid of said subject.

One preferred embodiment of the present application relates to PAM for use as a medicament in a subject identified as having a level of PAM and/or its isoforms and/or fragments thereof below a threshold and/or a peptide-Gly/peptide-amide ratio above a threshold in a sample of bodily fluid of said subject.

One embodiment of the present application relates to PAM for use as a medicament, wherein said use comprises testing a subject whether the subject has a level of PAM and/or its isoforms and/or fragments thereof below a threshold and/or a peptide-Gly/peptide-amide ratio above a threshold in a sample of bodily fluid of said subject, and providing treatment with PAM if the subject is identified as having risk for a disease or disorder.

One embodiment of the present application relates to PAM for use as a medicament, wherein PAM is combined with ascorbate and/or copper.

Subject-matter of the present application is also a pharmaceutical formulation comprising peptidylglycine alpha-amidating monooxygenase (PAM).

One embodiment of the present application relates to a pharmaceutical formulation comprising PAM, wherein said pharmaceutical formulation is administered orally (e.g. inhalation), epicutaneously, subcutaneously, intradermally, sublingually, intramuscularly, intraarterially, intravenously, or via the central nervous system (CNS, intracerebrally, intracerebroventricularly, intrathecally) or via intraperitoneal administration.

One preferred embodiment of the present application relates to a pharmaceutical formulation, wherein said pharmaceutical formulation is a solution, preferably a ready-to-use solution.

Another embodiment of the present application relates to a pharmaceutical formulation, wherein said pharmaceutical formulation is in a freeze-dried state.

Another embodiment of the present application relates to a pharmaceutical formulation, wherein said pharmaceutical formulation is administered intra-muscular.

Another specific embodiment of the present application relates to a pharmaceutical formulation, wherein said pharmaceutical formulation is administered intra-vascular.

Another preferred embodiment of the present application relates to a pharmaceutical formulation, wherein said pharmaceutical formulation is administered via infusion.

One embodiment of the present application relates to a pharmaceutical formulation, wherein said pharmaceutical formulation is to be administered systemically.

Another embodiment of the present application relates to a pharmaceutical formulation, the formulation comprising PAM and/or optionally one or more pharmaceutically acceptable ingredients

Another preferred embodiment of the present application relates to a pharmaceutical formulation, the formulation comprising PAM, ascorbate and/or copper.

Another embodiment of the present application relates to a pharmaceutical formulation, the formulation comprising PAM in combination with ascorbate and/or copper.

Subject-matter of the present application is also a method of treatment in a subject, the method comprising administering PAM to said subject, the method further comprising
i. reducing the potential or risk for a disease or disorder, and/or
ii. reducing the occurrence of a disease or disorder, and/or
iii. reducing the severity of a disease or disorder.

One embodiment of the present application relates to a method of treatment in a subject, wherein said disease or disorder is selected from the group comprising dementia, cardiovascular disorders, kidney diseases, cancer, inflammatory or infectious diseases and/or metabolic diseases.

Another embodiment of the present application relates to a method of treatment in a subject, wherein said subject is characterized by
a level of PAM and/or its isoforms and/or fragments thereof below a threshold and/or
a peptide-Gly/peptide-amide ratio above a threshold

in a sample of bodily fluid of said subject.

Another preferred embodiment of the present application relates to a method of treatment in a subject, wherein the level of PAM and/or its isoforms and/or fragments thereof is the total concentration of PAM and/or its isoforms and/or fragments thereof having at least 12 amino acids or the activity of PAM and/or its isoforms and/or fragments thereof.

One embodiment of the present application relates to a method of treatment in a subject, wherein the total concentration of PAM and/or its isoforms and/or fragments thereof having at least 12 amino acids or the activity of PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

Another embodiment of the present application relates to a method of treatment in a subject, wherein the sample of bodily fluid of said subject is selected from the group of blood, serum, plasma, urine, cerebrospinal fluid (CSF), and saliva.

Another preferred embodiment of the present application relates to a method of treatment in a subject, wherein said PAM is selected from the group comprising isolated and/or recombinant and/or chimeric PAM.

One preferred embodiment of the present application relates to peptidylglycine alpha-amidating monooxygenase (PAM) for use as a medicament for treatment of a subject, wherein the level of PAM and/or its isoforms and/or fragments thereof is the total concentration of PAM and/or its isoforms and/or fragments thereof having at least 12 amino acids or the activity of PAM and/or its isoforms and/or fragments thereof in a sample of bodily fluid of said subject.

One preferred embodiment of the present application relates to peptidylglycine alpha-amidating monooxygenase (PAM) for use as a medicament for treatment of a subject, wherein the activity of PAM and/or its isoforms and/or fragments thereof in a sample of bodily fluid in said subject is selected from the group comprising the sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

One preferred embodiment of the present application relates to peptidylglycine alpha-amidating monooxygenase (PAM) for use as a medicament for treatment of a subject, wherein the total concentration of PAM and/or its isoforms and/or fragments thereof having at least 12 amino acids in a sample of bodily fluid in said subject is detected with an immunoassay.

One preferred embodiment of the present application relates to peptidylglycine alpha-amidating monooxygenase (PAM) for use as a medicament for treatment of a subject, wherein the activity of PAM and/or its isoforms and/or fragments thereof in a sample of bodily fluid in said subject is detected using a peptide-Gly as substrate.

One preferred embodiment of the present application relates to peptidylglycine alpha-amidating monooxygenase (PAM) for use as a medicament for treatment of a subject, wherein the activity of PAM and/or its isoforms and/or fragments thereof in a sample of bodily fluid in said subject is detected using a peptide-Gly as substrate and wherein the peptide-Gly substrate is selected from the group comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin and vasopressin.

One preferred embodiment of the present application relates to peptidylglycine alpha-amidating monooxygenase (PAM) for use as a medicament for treatment of a subject, wherein the level of PAM and/or isoforms and/or fragments thereof in a bodily fluid sample of said subject is measured using an assay, wherein said assay is comprising two binders that bind to two different regions of PAM, wherein the two binders are directed to an epitope of at least 5 amino acids, preferably at least 4 amino acids in length, wherein said two binders are directed to an epitope comprised within the following sequences of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24) and recombinant PAM (SEQ ID No. 10).

One preferred embodiment of the present application relates to peptidylglycine alpha-amidating monooxygenase (PAM) for use as a medicament for treatment of a subject, wherein the method for determining the activity of PAM and/or isoforms or fragments thereof in a bodily fluid sample of a subject is comprising the steps:
contacting said sample with a capture-binder that binds specifically to active full-length PAM, its isoforms and/or active fragments thereof,
separating PAM bound to said capture-binder
adding a substrate of PAM to said separated PAM

quantifying PAM activity by measuring the conversion of the substrate of PAM

One preferred embodiment of the present application relates to peptidylglycine alpha-amidating monooxygenase (PAM) for use as a medicament for treatment of a subject,

wherein the method for determining the activity of PAM and/or isoforms and/or fragments thereof in a bodily fluid sample of a subject is comprising the steps:
contacting said sample with a substrate (peptide-Gly) of PAM for an interval of time at t=0 min and t=n+1 min
detecting the reaction product (alpha-amidated peptide) of PAM in said sample at t=0 min and t=n+1 min, and
quantifying the activity of PAM by calculating the difference of the reaction product between t=0 and t=n+1.

One preferred embodiment of the present application relates to peptidylglycine alpha-amidating monooxygenase (PAM) for use as a medicament for treatment of a subject,

wherein said binders are selected from the group comprising an antibody, an antibody fragment or a non-Ig scaffold.

One preferred embodiment of the present application relates to peptidylglycine alpha-amidating monooxygenase (PAM) for use as a medicament for treatment of a subject,

wherein said subject is characterized by
a level of PAM and/or its isoforms and/or fragments thereof below a threshold and/or
a peptide-Gly/peptide-amide ratio above a threshold

in a bodily fluid of said subject.

One preferred embodiment of the present application relates to peptidylglycine alpha-amidating monooxygenase (PAM) for use as a medicament for treatment of a subject,

wherein said subject is characterized by
an ADM-Gly/bio-ADM ratio above a threshold and/or
a bio-ADM concentration below a threshold

in a bodily fluid of said patient.

The threshold is pre-determined by measuring the level of PAM and/or its isoforms and/or fragments and/or ADM-NH₂ thereof in healthy controls and calculating e.g., the according 25-percentile, more preferably the 10-percentile, even more preferably the 5-percentile. The lower boarder of the 25-percentile, more preferably the 10-percentile, even more preferably the 5-percentile, defines the threshold for healthy versus diseased patients or healthy versus subjects at risk of getting a disease or subjects not at risk of getting an adverse event versus subjects at risk of getting an adverse event, if the level of said diseased subjects or subjects at risk of getting a disease or adverse event is below a threshold. The level of PAM and/or its isoforms and/or fragments thereof may be detected as total PAM concentration and/or PAM activity. In relation to said percentiles, the lower threshold that divides between healthy and diseased patients or healthy versus subjects at risk of getting a disease or subjects not at risk of getting an adverse event versus subjects at risk of getting an adverse event by detecting the PAM activity in plasma may be between 15 and 8 μg/(L*h) or below, more preferably between 13.5 and 8 μg/(L*h) or below, even more preferred between 10.5 and 8 μg/(L*h) or below, most preferred below 8 μg/(L*h); PAM activity in serum may be between 10 and 5 μg/(L*h) or below, more preferably between 8 and 5 μg/(L*h) or below, most preferred below 5 μg/(L*h) using a PAM activity assay.

In relation to said percentiles, the lower threshold that divides between healthy and diseased patients or healthy versus subjects at risk of getting a disease or subjects not at risk of getting an adverse event versus subjects at risk of getting an adverse event by detecting ADM-NH₂ is equal or below 15 pg/ml, preferably equal or below 10 pg/ml, preferably equal or below 5 pg/mL.

The threshold is pre-determined by measuring the ratio of ADM-Gly to ADM-NH₂ in healthy controls and calculating e.g., the according 75-percentile, more preferably the 90-percentile, even more preferably the 95-percentile. The upper boarder of the 75-percentile, more preferably the 90-percentile, even more preferably the 95-percentile, defines the threshold for healthy versus diseased patients or healthy versus subjects at risk of getting a disease or subjects not at risk of getting an adverse event versus subjects at risk of getting an adverse event, if the level of said diseased subjects or subjects at risk of getting a disease or adverse event is above a threshold. In relation to said percentiles, the upper threshold that divides between healthy and diseased patients or healthy versus subjects at risk of getting a disease or subjects not at risk of getting an adverse event versus subjects at risk of getting an adverse event by detecting the ration of ADM-Gly to ADM-NH₂, wherein the ADM-Gly/ADM-NH₂ ratio is in a range between 1 and 10, preferably between 1.5 and 7.5, preferably between 2 and 5, most preferred the threshold is 2.5.

The predetermined value can vary among particular populations selected, depending on certain factors, such as gender, age, genetics, habits, ethnicity or alike.

The person skilled in the art knows how to determine thresholds from conducted previous studies. The person skilled in the art knows that a specific threshold value may depend on the cohort used for calculating a pre-determined threshold that can be later-on used in routine. The person skilled in the art knows that a specific threshold value may depend on the calibration used in the assay. The person skilled in the art knows that a specific threshold value may depend on the sensitivity and/or specificity that seems to be acceptable for the practitioner.

The sensitivity and specificity of a diagnostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e., apparently healthy) and “disease” populations (i.e., patients suffering from an infection). Depending on the particular diagnostic question to be addressed, the reference group must not be necessarily “normal”, but it might be a group of patients suffering from another disease, from which the diseased group of interest shall be differentiated. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a disease. ROC curves can be used even when test results do not necessarily give an accurate number. As long as one can rank results, one can create a ROC curve. For example, results of a test on “disease” samples might be ranked according to degree (e.g, 1=low, 2=normal, and 3=high). This ranking can be correlated to results in the “normal” population, and a ROC curve created. These methods are well known in the art (see, e.g., Hartley et al, 1982). Preferably, a threshold is selected to provide a ROC curve area of greater than about 0.5, more preferably greater than about 0.7. The term “about” in this context refers to +/−5% of a given measurement.

Once the threshold value is determined by using a previous study cohort and taking into consideration all the above-mentioned points the medical practitioner will use the pre-determined threshold for diagnosing or prognosing a disease and/or predicting a risk of getting a disease or an adverse event in a subject and will determine whether the subject has a value above or below said pre-determined threshold value in order to make an appropriate diagnosis, prognosis, prediction or monitoring.

The mentioned threshold values above might be different in other assays, if these have been calibrated differently from the assay system used in the present invention. Therefore, the mentioned threshold(s) shall apply for such differently calibrated assays accordingly, taking into account the differences in calibration. One possibility of quantifying the difference in calibration is a method comparison analysis (correlation) of the assay in question (e.g. PAM assay) with the respective biomarker assay used in the present invention by measuring the respective biomarker or it's activity (e.g. PAM) in samples using both methods. Another possibility is to determine with the assay in question, given this test has sufficient analytical sensitivity, the median biomarker level of a representative normal population, compare results with the median biomarker levels with another assay and recalculate the calibration based on the difference obtained by this comparison. With the calibration used in the present invention, samples from normal (healthy) subjects have been measured: the median plasma PAM activity was 18.4 μg/(L*h) (inter quartile range [IQR] 13.5-21.9 μg/(L*h)), the median serum PAM activity was 11.0 μg/(L*h) (inter quartile range [IQR] 8.1-13.1 μg/(L*h). In samples from normal (healthy) subjects have ADM-NH₂ been measured: median plasma bio-ADM (mature ADM-NH₂) was 13.7 pg/ml (inter quartile range [IQR] 9.6-18.7 pg/mL) (Weber et al. 2017. JALM, 2(2): 222-233).

One preferred embodiment of the present application relates to peptidylglycine alpha-amidating monooxygenase (PAM) for use as a therapy in a subject,

wherein said subject is a healthy subject that has been predicted as having an increased risk to develop a disease or disorder.

Another preferred embodiment of the present application relates to peptidylglycine alpha-amidating monooxygenase (PAM) for use as a therapy in a subject,

wherein said subject is a healthy subject that has been predicted as having an increased risk to develop a disease or disorder or an adverse event in the future.

The term “PAM” of the present disclosure refers to isolated PAM, including splice variants, isoforms, and polymorphic forms thereof. Also included are recombinant PAM (RecPAM) and chimeric PAM. In specific aspects, the PAM is RecPAM. The amino acid sequence of PAM isoform 1 to 6 is shown in SEQ ID No. 1 to 6. In some aspects, PAM disclosed herein has at least about 70%, at least about 75%, at least about 80%, at least about 85%, at least about 90%, at least about 95%, at least about 96%, at least about 97%, at least about 98%, or at least about 99% sequence identity to the amino acid sequence of SEQ ID No.: 1 to 6.

It is to be understood by the skilled artisan, that the PAM isoform sequences (SEQ ID No. 1 to 6) as represented in the sequence list, contain an N-terminal signal sequence (amino acid 1-20). This N-terminal signal sequence is cleaved off prior to secretion of the protein. Therefore, in a preferred embodiment the PAM isoform sequences (SEQ ID No. 1 to 6) and/or fragments thereof do not contain the N-terminal signal sequence.

In some aspects, the PAM is a functional fragment (i.e., PHM (SEQ ID No. 7) and PAL (SEQ ID No. 8), PAM conserving at least about 10%, at least about 20%, at least about 30%, at least about 40%, at least about 50%, at least about 60%, at least 70%, at least about 80%, or at least about 90% of the PAM activity of the corresponding functional fragment of PAM. In some aspects, the PAM is a variant or a derivative of PAM disclosed herein.

The percentage of identity of an amino acid or nucleic acid sequence, or the term “% sequence identity”, is defined herein as the percentage of residues in a candidate amino acid or nucleic acid sequence that is identical with the residues in a reference sequence after aligning the two sequences and introducing gaps, if necessary, to achieve the maximum percent identity. In a preferred embodiment, the calculation of said at least percentage of sequence identity is carried out without introducing gaps. Methods and computer programs for the alignment are well known in the art, for example “Align 2” or the BLAST service of the National Center for Biotechnology Information (NCBI).

PAM for use according to the present disclosure can be a commercial PAM enzyme, or any composition comprising the PAM enzyme and any means capable of producing a functional PAM enzyme in the context of the current invention, such as DNA or RNA nucleic acids encoding a PAM protein. The nucleic acid encoding PAM may be embedded in suitable vectors such as plasmids, phagemids, phages, (retro)viruses, transposons, gene therapy vectors and other vectors capable of inducing or conferring production of PAM. Also native or recombinant microorganisms, such as bacteria, fungi, protozoa and yeast may be applied as a source of PAM in the context of the current disclosure.

In some aspects, the mammalian PAM is a human or a bovine PAM.

As used herein the terms “treat”, “treatment”, “treatment of”, “therapy” or “therapy of” refers to (i) reducing the potential or risk for a disease or disorder, e.g. dementia, cardiovascular disorders, kidney diseases, cancer, inflammatory or infectious diseases and/or metabolic diseases, (ii) reducing the occurrence of a disease or disorder, e.g. dementia, cardiovascular disorders, kidney diseases, cancer, inflammatory or infectious diseases and/or metabolic diseases, (iii) reducing the severity (e.g., ameliorating the symptoms) of a disease or disorder, e.g. dementia, cardiovascular disorders, kidney diseases, cancer, inflammatory or infectious diseases and/or metabolic diseases, or (iv) a combination thereof.

The terms “subject” or “patient” as used herein refer to any subject, particularly a mammalian subject, for whom therapy or prognosis of a disease is desired. As used herein, the terms “subject” or “patient” include any human or nonhuman animal. As used herein, phrases such as “a patient having a disease” includes subjects, such as mammalian subjects, that would benefit from the administration of a therapy with PAM, as disclosed herein.

In some aspects of the present disclosure, a therapeutic agent for the treatment or prevention a disease, e.g., dementia, cardiovascular disorders, kidney diseases, cancer, inflammatory or infectious diseases and/or metabolic diseases, can comprise PAM, e.g., RecPAM or isolated PAM; alone or in combination with one or more standard therapeutic agents generally used for the treatment or prevention of a disease, e.g., dementia, cardiovascular disorders, kidney diseases, cancer, inflammatory or infectious diseases and/or metabolic diseases.

“Therapeutically effective amount” means level or amount of therapeutic agent that is aimed at, without causing significant negative or adverse side effects to the target, (1) delaying or preventing the onset of the target disease, disorder, or condition; (2) slowing down or stopping the progression, aggravation, or deterioration of one or more symptoms of the target disease, disorder, or condition; (3) bringing about ameliorations of the symptoms of the target disease, disorder, or condition; (4) reducing the severity or incidence of the target disease, disorder, or condition; or (5) curing the target disease, disorder, or condition. A therapeutically effective amount may be administered prior to the onset of the target disease, disorder, or condition, for a prophylactic or preventive action. Alternatively, or additionally, the therapeutically effective amount may be administered after initiation of the target disease, disorder, or condition, for a therapeutic action.

In some aspects, the PAM (e.g., RecPAM) is administered as doses of at least about 500 U/kg, at least about 600 U/kg, at least about 700 U/kg, at least about 800 U/kg, at least about 900 U/kg, at least about 1000 U/kg, at least about 1100 U/kg, at least about 1200 U/kg, at least about 1300 U/kg, at least about 1400 U/kg, at least about 1500 U/kg, at least about 1600 U/kg. at least about 1700 U/kg, at least about 1800 U/kg, at least about 1900 U/kg. or at least about 2000 U/kg per dose. In some aspects, the PAM (e.g. RecPAM) is administered as doses above 2000 U/kg per dose. In some aspects, the PAM (e.g., RecPAM) is administered as doses below 500 U/kg per dose.

In some aspects, the PAM (e.g., RecPAM) is administered at a dose between about 500 U/kg and about 1500 U/kg, between about 600 U/kg and about 1400 U/kg, between about 700 U/kg and about 1300 U/kg, between about 800 U/kg and about 1200 U/kg, or between about 900 U/kg and about 1100 U/kg. In some specific aspects, PAM is administered as about 1000 U/kg doses.

In some aspects, the PAM is a human or bovine PAM. In some aspects, the PAM is a recombinant PAM (RecPAM). In some aspects, the PAM is a chimeric PAM. In a particular aspect, the chimeric PAM is RecPAM (SEQ ID NO: 1, 2, 3, 4, 5, 6, 7, 8). In some aspects, PAM disclosed herein has at least about 70%, at least about 75%, at least about 80%, at least about 85%, at least about 90%, at least about 95%, at least about 96%, at least about 97%, at least about 98%, or at least about 99% sequence identity to the amino acid sequence of SEQ ID NO: 1, 2, 3, 4, 5, 6, 7, 8.

In some aspects, the PAM is a functional fragment (i.e., PHM (SEQ ID No. 7) and PAL (SEQ ID No. 8), (i.e., a fragment of the PAM, e.g., PAM conserving at least about 10%, at least about 20%, at least about 30%, at least 40%, at least about 50%, at least about 60%, at least 70%, at least about 80%, or at least about 90% of the PAM activity of the corresponding full-length PAM). In some aspects, the PAM is a variant or a derivative of a PAM disclosed herein.

PAM is administered at a dose that significantly increases the concentration of bio-ADM in the blood of a patient. A significant increase in the concentration of bio-ADM is defined as an increase of about 10%, more preferred of about 25%, even more preferred of about 50%, even more preferred of about 100%, even more preferred of about 200%, even more preferred of about 300%, even more preferred of about 500%, most preferred up to 1000%. It is preferred that the concentration of bio-ADM is increased to the median concentration of a healthy population. Samples from normal (healthy) subjects have been measured: median plasma bio-ADM (mature ADM-NH₂) was 24.7 pg/ml, the lowest value 11 pg/ml and the 99^{th} percentile 43 pg/ml (Marino et al. 2014. Critical Care 18:R34).”

In some aspects, the PAM is RecPAM and it is administered at a dose of at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 1.1 mg/kg, at least about 1.3 mg/kg, at least about 1.4 mg/kg, at least about 1.5 mg/kg, at least about 1.6 mg/kg, at least about 1.7 mg/kg, at least about 1.8 mg/kg, at least about 1.9 mg/kg, at least about 2 mg/kg, at least about 2.1 mg/kg, at least about 2.2 mg/kg, at least about 2.3 mg/kg, or at least about 2.4/kg per dose. In some aspects, the PAM is administered as doses above 2.4 mg/kg per dose.

In some aspects, the PAM is RecPAM, and it is administered at a dose of at least about 100 U/kg, at least about 200 U/kg, at least about 300 U/kg. at least about 400 U/kg, at least about 500 U/kg, at least about 600 U/kg, at least about 700 U/kg, at least about 800 U/kg, at least about 900 U/kg, at least about 1000 U/kg, at least about 1100 U/kg, at least about 1200 U/kg, at least about 1300 U/kg, at least about 1400 U/kg, at least about 1500 U/kg, at least about 1600 U/kg, at least about 1700 U/kg, at least about 1800 U/kg, at least about 1900 U/kg, or at least about 2000 U/kg. The PAM is RecPAM, and it is administered at a dose below 100 U/kg, the PAM is RecPAM, and it is administered at a dose above 2000 U/kg.

In some aspects, the PAM is RecPAM and it is administered at a dose between about 0.8 mg/kg and about 2.4 mg/kg, between about 0.9 mg/kg and about 2.3 mg/kg, between about 1 mg/kg and about 2.2 mg/kg, between about 1.1 mg/kg and about 2.1 mg/kg, between about 1.2 mg/kg and about 2 mg/kg, between about 1.3 mg/kg and about 1.9 mg/kg, between about 1.4 mg/kg and about 1.8 mg/kg, or between about 1.5 mg/kg and about 1.7 mg/kg. In some specific aspects, PAM is administered as about 1.6 mg/kg doses.

In some aspects, the PAM is RecPAM and it has a specific activity of at least about 100 U/mg, at least about 200 U/mg, at least about 300 U/mg, at least about 400 U/mg, at least about 500 U/mg, at least about 600 U/mg, at least about 700 U/mg, at least about 800 U/mg, at least about 900 U/mg, at least about 1000 U/mg, at least about 1100 U/mg, at least about 1200 U/mg, at least about 1300 U/mg, at least about 1400 U/mg, at least about 1500 U/mg, at least about 1600 U/mg, at least about 1700 U/mg, at least about 1800 U/mg, at least about 1900 U/mg, or at least about 2000 U/mg.

In some aspects, the PAM is RecPAM and it has a specific activity of about 1000 U/mg. In some aspects, the PAM is RecPAM and it has a specific activity between about 600 U/mg and about 700 U/mg, or between about 500 U/mg and about 800 U/mg, or between about 400 U/mg and about 900 U/mg, or between about 300 U/mg and about 1000 U/mg, or between about 200 U/mg and about 1100 U/mg, or between 100 U/mg and about 1200 U/mg. In some aspects, the PAM is RecPAM and it has a specific activity below 100 U/mg. In some aspects, the PAM is RecPAM and it has a specific activity above 1200 U/mg.

In some aspects, only one dose of PAM (e.g., RecPAM) is administered per treatment (e.g., one dose per day for 1-7 days). In other aspects, more than one dose of PAM is administered. In some aspects, two, three, four, five, six, seven, eight, nine, 10, 11, 12, 13, 14, 15, 16, 17, 18, 19, 20, or 21 doses of PAM are administered (e.g., at least two doses per day for 1-7 days).

In some aspects, the PAM doses are administered daily. In other aspects, PAM doses are administered every 2, 3, 4, 5, 6 or 7 days.

In some aspects, a single dose is administered every day. In some aspects, 2, 3, or more doses are administered every day.

In some aspects, the treatment with PAM is less than about 7 days. In some aspects, the treatment with PAM is less than 6 days, less than 5 days, less than 4 days, less than 3 days, less than 2 days, or less than 1 day.

In some aspects the treatment of PAM is more than 7 days, more than 14 days, more than 21 days, more than 28 days, more than 1 month, more than 3 months, more than 6 months, more than 12 months, more than 2 years, more than 5 years or more than 10 years.

In some aspects, the second measurement is conducted 1, 2, 3, 4, 5, 6, 7, 8, 9, 10, 11, 12, 13, 14, 15, 16, 17, 18, 19, 20, 21, 22, 23, 24, 25, 26, 27, or 28 days, or at 1, 2, 3, 4, 5, 6, 7, 8, 9, 10, 11, 12, 13, 14, 15 or 16 weeks, or at intervening times, after administering the PAM, e.g., RecPAM.

The formulation, dosage regimen, and route of administration of a PAM, e.g., RecPAM, can be adjusted to provide an effective amount for an optimum therapeutic response according to the method disclosed herein. With regard to the administration of PAM, the PAM may be administered through any suitable means, compositions and routes known in the art. With regard to dosage regiments, a single bolus can be administered, several divided doses can be administered over time or the dose can be proportionally reduced or increased as indicated by the exigencies of the therapeutic situation.

The present disclosure provides a method of determining whether to treat a patient having a disease or preventing a disease with a therapeutic regimen comprising the administration of PAM, wherein the method comprises:
(a) measuring
   the level of PAM and/or its isoforms and/or fragments thereof and/or
   the peptide-Gly/peptide-amide ratio
   in a bodily fluid of said patient, and
(b) treating the patient, or suspending the treatment with a therapeutic regimen comprising the administration of PAM, e.g., isolated or recombinant PAM, if the patient is determined to have higher or lower concentrations or activities in the sample compared to a predetermined threshold level or levels, or compared to the level or levels in one or more controls.

The peptide-Gly may be selected from the group comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin, vasopressin.

In a preferred embodiment said peptide-Gly is ADM-Gly and said peptide-amide is ADM-NH₂.

As used herein, the term diseases or disorders include all “PAM-related” diseases or disorders that are known now, or that will be found in the future, to be associated with a decrease in PAM activity and/or increase in the peptide-Gly/peptide-amide ratio.

In a specific embodiment of said disease or disorder is selected from the group comprising:
dementia, wherein said dementia is selected from the group comprising mild cognitive impairment (MCI), Alzheimer's disease, vascular dementia, mixed Alzheimer's disease and vascular dementia, Lewy body dementia, frontotemporal dementia, focal dementias (including progressive aphasia), subcortical dementias (including Parkinson's disease) and secondary causes of dementia syndrome (including intracranial lesions).
cardiovascular disorders, wherein said cardiovascular disorders may be selected from a group comprising atherosclerosis, hypertension, heart failure (including acute and acute decompensated heart failure), atrial fibrillation, cardiovascular ischemia, cerebral ischemic injury, cardiogenic shock, stroke (including ischemic and hemorrhagic stroke and transient ischemic attack) and myocardial infarction,
kidney diseases, wherein said kidney diseases may be selected from a group comprising renal toxicity (drug-induced kidney disease), acute kidney injury (AKI), chronic kidney disease (CKD), diabetic nephropathy, end-stage renal disease (ESRD),
cancer, wherein said cancer may be selected from a group comprising prostate cancer, breast cancer, lung cancer, colorectal cancer, bladder cancer, ovarian cancer, cervical cancer, skin cancer (including melanoma), stomach cancer, liver cancer, pancreatic cancer, leukemia, non-hodgkin's lymphoma, kidney cancer, esophagus cancer, pharyngeal cancer,
infectious diseases caused by infectious organisms such as bacteria, viruses, fungi or parasites, said infectious disease is selected from the group comprising SIRS, sepsis, and septic shock.
metabolic diseases selected from the group comprising diabetes type 1, diabetes type 2, metabolic syndrome.

In one embodiment of the present application said disease is dementia and said dementia is selected from the group comprising mild cognitive impairment (MCI), Alzheimer's disease, vascular dementia, mixed Alzheimer's disease and vascular dementia, Lewy body dementia, frontotemporal dementia, focal dementias (including progressive aphasia), subcortical dementias (including Parkinson's disease) and secondary causes of dementia syndrome (including intracranial lesions).

In a specific embodiment said dementia is Alzheimer's disease.

In one embodiment of the present application said disease is cancer and said cancer is selected from the group comprising prostate cancer, breast cancer, lung cancer, colorectal cancer, bladder cancer, ovarian cancer, cervical cancer, skin cancer (including melanoma), stomach cancer, liver cancer, leukemia, non-hodgkin's lymphoma, kidney cancer, esophagus cancer and pharyngeal cancer.

In a specific embodiment said cancer is colorectal cancer.

In one embodiment of the present application said disease is a cardiovascular disorder, wherein said cardiovascular disorder is selected from a group comprising atherosclerosis, hypertension, heart failure (including acute and acute decompensated heart failure), atrial fibrillation, cardiovascular ischemia, cerebral ischemic injury, cardiogenic shock, stroke (including ischemic and hemorrhagic stroke and transient ischemic attack) and myocardial infarction.

In a specific embodiment said cardiovascular disorder is heart failure (including acute and acute decompensated heart failure).

In another specific embodiment said cardiovascular disorder is stroke stroke (including ischemic and haemorrhagic stroke and transient ischemic attack) and myocardial infarction.

In another specific embodiment said cardiovascular disorder is atrial fibrillation.

In another specific embodiment of the present application said disease is SIRS, sepsis or septic shock.

In another specific embodiment of the present application said disease is diabetes type 1, diabetes type 2, metabolic syndrome.

In one embodiment, the patients with “PAM-related” disease or disorder is a patient with chronically reduced bio-ADM concentrations which may suffer from a subclinical endothelial dysfunction. Subclinical endothelial dysfunction e.g., in the brain may lead to dementia especially Alzheimer's disease.

The bodily fluid in the context of the present invention maybe selected from the group of blood, serum, plasma, cerebrospinal fluid (CSF), urine, saliva, sputum, and pleural effusions. In a specific embodiment of said method said sample is selected from the group comprising whole blood, serum and plasma.

The term “pharmaceutical formulation” as used herein refers to a preparation which is in such form as to permit the biological activity of an active ingredient contained therein to be effective, and which contains no additional components which are unacceptably toxic to a subject to which the formulation would be administered.

The present invention also relates to a pharmaceutical formulation comprising a therapeutically effective dose of PAM, e.g., RecPAM, in combination with at least one pharmaceutically acceptable excipient.

“Pharmaceutically acceptable excipient” refers to an excipient that does not produce an adverse, allergic or other untoward reaction when administered to a subject. It includes in addition to a therapeutic protein, carriers, various diluents, fillers, salts, buffers, stabilizers, solubilizers, and other materials well known in the art. The characteristics of the carrier will depend on the route of administration.

In one embodiment of the present invention said pharmaceutical formulation is administered orally (e.g., inhalation), epicutaneously, subcutaneously, intradermally, sublingually, intramuscularly, intraarterially, intravenously, or via the central nervous system (CNS, intracerebrally, intracerebroventricularly, intrathecally) or via intraperitoneal administration.

For administration by inhalation, the compound is delivered in the form of an aerosol spray from a pressurized container or dispenser which contains a suitable propellant, e.g., a gas such as carbon dioxide or a nebulizer.

Subject matter of the present invention is a pharmaceutical formulation of PAM for use in therapy of a subject according to the present invention, wherein said pharmaceutical formulation is a solution, preferably a ready-to-use solution.

Subject matter of the present invention is a pharmaceutical formulation of PAM for use in therapy of a subject according to the present invention, wherein said pharmaceutical composition is in a freeze-dried state.

Subject matter of the present invention is a pharmaceutical formulation for use in therapy of a subject, wherein said pharmaceutical formulation is administered via infusion.

Subject matter of the present invention is a pharmaceutical formulation for use in therapy of a subject, wherein said pharmaceutical formulation is to be administered systemically.

Therapeutic proteins for subcutaneous administration are frequently administered at high-concentrations. Particularly contemplated high-concentrations of therapeutic proteins (without taking into account the weight of chemical modifications such as PEGylation), are at least about 70, 80, 90, 95, 100, 105, 110, 115, 120, 125, 130, 135, 140, 145, 150, 155, 160, 165, 175, 180, 185, 190, 195, 200, 250, 300, 350, 400, 450, or 500 mg/ml, and/or less than about 250, 300, 350, 400, 450 or 500 mg/ml. Exemplary high-concentrations of therapeutic proteins, such as enzymes, in the formulation may range from about 100 mg/ml to about 500 mg/ml. Preferably, the concentrations of the therapeutic protein according to the invention are in the range of about 100-300 mg/ml, more preferred in the range of 135-165 mg/ml, most preferred of about 150 mg/ml. A further most preferred concentration is about 100 mg/ml. In this context a concentration of “about” a given value, e.g. the upper or lower limit of a given concentration range, is to be understood as encompassing all concentration deviating up to +10% from this given value.

Chemical modifications may be employed for protecting PAM from degradation, extending in vivo half-life, providing prolonged drug release, augmenting drug efficacy, while reducing side effects, reducing administration frequency and lowering drug dosage. Other advantages include alleviation of pain associated with frequent injections and significant reduction in cost of treatment. Chemical modifications include covalent conjugation of polymers such as PEG (polyethyleneglycol), polysialic acid, hyperglycosylation and mannosylation (Patel et al. 2014. Ther. Deliv. 5(3): 337-365). Alternative formulation approaches include colloidal carriers as protein delivery systems such as microparticles, nanoparticles, liposomes, carbon nanotubes and micelles (Patel et al. 2014. Ther. Deliv. 5(3): 337-365).

In one embodiment of the invention said covalently conjugated polymer is selected from the group of branched or unbranched polyethyleneglycol (PEG), branched or unbranched polypropyleneglycol (PPG) hydroxyethyl starch (HES) or a derivative thereof, polysialic acids (PSAs) or a derivative thereof, or a glycine-rich homo-amino-acid polymer (HAP) (see WO2020254197 for reference). The polymer can be of any molecular weight.

In another aspect of the invention PAM may be administered via gene therapy. There are two major conventional methods of gene therapy, the first is to use viral capsids to encapsulate a DNA sequence of interest for introduction into mammalian tissue. Such viral capsids are generally termed viral vectors, and a variety of vectors have been used including HIV and adenoviruses. Such viral vectors are generally constructed so that they should not reproduce in-vivo, and usually contain a reverse transcriptase that results in splicing of the viral vector borne DNA into the host genome. The second major conventional method of gene therapy, DNA gene therapy, uses the much simpler method of injection of DNA into the patient. This introduces considerably less packaging material into the host, and DNA constructs are generally smaller. DNA is not incorporated into the host genome, but is instead maintained separately in circlets either inside the nucleus or at the nuclear wall inside the cell. These circlets may become associated with histones within the nucleus.

In a specific embodiment of the application, an assay is used for determining the level of PAM and/or its isoforms and/or fragments thereof, wherein such assay is a sandwich assay, preferably a fully automated assay.

In one embodiment of the application it may be a so-called POC-test (point-of-care) that is a test technology, which allows performing the test within less than 1 hour near the patient without the requirement of a fully automated assay system. One example for this technology is the immunochromatographic test technology.

In one embodiment of the application such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®, Ortho Clinical Diagnostics Vitros®.

In a specific embodiment of the application, at least one of said two binders is labeled in order to be detected.

The preferred detection methods comprise immunoassays in various formats such as for instance radioimmunoassay (RIA), homogeneous enzyme-multiplied immunoassays (EMIT), chemiluminescence- and fluorescence-immunoassays, Enzyme-linked immunoassays (ELISA), Luminex-based bead arrays, protein microarray assays, and rapid test formats such as for instance immunochromatographic strip tests.

In a preferred embodiment, said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005); Hultschig et al. 2006 Curr Opin Chem Biol. 10 (1):4-10).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type. In the context of the present invention, fluorescence based assays comprise the use of dyes, which may for instance be selected from the group comprising FAM (5- or 6-carboxyfluorescein), VIC, NED, fluorescein, fluorescein-isothiocyanate (FITC), IRD-700/800, Cyanine dyes, such as CY3, CY5, CY3.5, CY5.5, Cy7, xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA),

6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, coumarines such as umbelliferone, benzimides, such as Hoechst 33258; Phenanthridines, such as Texas Red, Yakima Yellow, Alexa Fluor, PET, ethidiumbromide, acridinium dyes, carbazol dyes, Phenoxazine dyes, porphyrine dyes, polymethine dyes, and the like.

In the context of the application, chemiluminescence based assays comprise the use of dyes, based on the physical principles described for chemiluminescent materials in (Kirk-Othmer, Encyclopedia of chemical technology, 4th ed. 1993. John Wiley & Sons, Vol. 15: 518-562, incorporated herein by reference, including citations on pages 551-562). Preferred chemiluminescent dyes are acridinium esters.

As mentioned herein, an “assay” or “diagnostic assay” can be of any type applied in the field of diagnostics. Such an assay may be based on the binding of an analyte to be detected to one or more capture probes with a certain affinity. Binders that may be used for determining the level of PAM and/or its isoforms and/or fragments thereof exhibit an affinity constant to PAM and/or its isoforms and/or fragments thereof of at least 10⁷ M⁻¹, preferred 108 M⁻¹, preferred affinity constant is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

In the context of the present application, “binder molecules” are molecules which may be used to bind target molecules or molecules of interest, i.e. analytes (i.e. in the context of the present invention PAM and its isoforms and fragments thereof), from a sample. Binder molecules have thus to be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain.

In a specific embodiment said binder may be selected from the group of antibody, antibody fragment or non-IgG scaffold.

Chemiluminescent label may be acridinium ester label, steroid labels involving isoluminol labels and the like.

Enzyme labels may be lactate dehydrogenase (LDH), creatine kinase (CPK), alkaline phosphatase, aspartate aminotransferase (AST), alanine aminotransferase (ALT), acid phosphatase, glucose-6-phosphate dehydrogenase and so on.

In one embodiment of the application at least one of said two binders is bound to a solid phase as magnetic particles, and polystyrene surfaces.

Subject matter of the application is a method for determining the level of PAM and/or isoforms and/or fragments thereof in a bodily fluid sample using an assay, wherein said assay is comprising two binders that bind to two different epitopes of PAM, wherein the two binders are directed to an epitope of at least 5 amino acids, preferably at least 4 amino acids in length.

An epitope, also known as antigenic determinant, is the part of an antigen (e.g. peptide or protein) that is recognized by the immune system, specifically by antibodies. For example, the epitope is the specific piece of the antigen to which an antibody binds. The part of an antibody that binds to the epitope is called a paratope. The epitopes of protein antigens are divided into two categories: conformational epitopes and linear epitopes, based on their structure and interaction with the paratope.

A linear or a sequential epitope is an epitope that is recognized by antibodies by its linear sequence of amino acids, or primary structure and is formed by the 3-D conformation adopted by the interaction of contiguous amino acid residues. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontiguous amino acid residues.

In one embodiment of the application linear epitopes are related to following sequences of immunization peptides of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24).

In one embodiment of the application, linear and/or conformational epitopes are related to the following sequences of PAM: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 10.

Said epitope may comprise at least 6 amino acids, preferably at least 5 amino acids, most preferred at least 4 amino acids.

In one embodiment of the application said first and second binder binds to an epitope comprised within the following sequences of PAM: SEQ ID No. 1, SEQ ID No. 2, SEQ ID No. 3, SEQ ID No. 4, SEQ ID No. 5 and SEQ ID No. 6.

In one embodiment of the application said first and second binder binds to an epitope comprised within the PAL subunit of PAM (SEQ ID No. 8).

In one embodiment of the application said first and second binder binds to an epitope comprised within the PHM subunit of PAM (SEQ ID No. 7).

In one specific embodiment of the application said first binder binds to an epitope comprised within the PAL subunit of PAM (SEQ ID No. 8) and said second binder binds to an epitope comprised within the PHM subunit of PAM (SEQ ID No. 7).

In one specific embodiment of the application said first and second binder binds to an epitope comprised within the following sequences of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24) and recombinant PAM (SEQ ID No. 10).

Use of at least two binders for the determination of the level of PAM and/or its isoforms and/or fragments thereof, wherein said at least one binder is directed to an epitope comprised within the following sequences of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24) and recombinant PAM (SEQ ID No. 10).

Subject of the present application is a method for determining the activity of PAM and/or isoforms and/or fragments thereof in a bodily fluid sample of a subject comprising the steps
Contacting said sample with a capture-binder that binds specifically to active full-length PAM, its isoforms and/or active fragments thereof,
Separating PAM bound to said capture-binder
Adding a substrate of PAM to said separated PAM
Quantifying PAM activity by measuring the conversion of the substrate of PAM.

In a specific embodiment of the present application said method is an enzyme capture assay (ECA, see e.g. U.S. Pat. Nos. 5,612,186A, 5,601,986A).

In a specific embodiment of said method for determining PAM activity in a bodily fluid sample of a subject said separation step is a washing step that removes ingredients of the sample that are not bound to said capture-binder from the captured PAM and/or its isoforms and/or fragments thereof. That separation step can be any other step that separates PAM bound to said capture-binder from the ingredients of said bodily fluid sample.

One embodiment of the present application involves a chemical assay for PAM. The assay uses a peptide substrate which reacts with PAM and/or its isoforms and/or fragments thereof to form a detectable reaction product. Alternatively, the rate of the reaction of the substrate can be monitored to determine the level of PAM and/or its isoforms and/or fragments thereof in a test sample.

Assays embodying such reagents and reactions can be performed in any suitable reaction vessel, for example, a test tube or well of a microtiter plate. Alternatively, assay devices may be developed in disposable form such as dipstick or test strip device formats which are well known to those skilled-in-the-art and which provide ease of manufacture and use. Such disposable assay devices may be packaged in the form of kits containing all necessary materials, reagents and instructions for use.

In an alternative assay embodiment, the rate at which the reaction occurs may be detected as an indication of the level of PAM and/or its isoforms and/or fragments thereof present in the test sample. For example, the rate at which the substrate is reacted may be used to indicate the level of PAM and/or its isoforms and/or fragments thereof present in the test sample. Alternatively, the rate at which the reaction product is formed may be used to indicate the level of PAM and/or its isoforms and/or fragments thereof present in the test sample.

In yet another embodiment, a capture or binding assay may be performed to determine the activity of PAM and/or its isoforms and/or fragments thereof. For example, an antibody reactive with PAM protein, but which does not interfere with its enzymatic activity, may be immobilized upon a solid phase. The test sample is passed over the immobile antibody, and PAM and/or its isoforms and/or fragments thereof, if present, binds to the antibody and is itself immobilized for detection. A substrate may then be added, and the reaction product may be detected to indicate the level of PAM and/or its isoforms and/or fragments thereof in the test sample. For the purposes of the present description, the term “solid phase” may be used to include any material or vessel in which or on which the assay may be performed and includes, but is not limited to, porous materials, nonporous materials, test tubes, wells, slides, etc.

In a specific embodiment of said method for the diagnosis or prognosis of a disease in a subject and/or predicting a risk of getting a disease or adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject said capture binder is immobilized on a surface. For the determination of PAM activity, a binder reactive with PAM and/or its isoforms and/or fragments thereof, but which does not interfere with enzymatic activity by more than 50%, preferably less than 40%, preferably less than 30%, may be immobilized upon a solid phase. To prevent inhibition of PAM the capture-binder should not bind PAM in the area around the active center and substrate binding region.

In a specific embodiment of said method for determining the level of PAM and/or its isoforms and/or fragments thereof in a bodily fluid sample of a subject said binder may be selected from the group of antibodies, antibody fragments, non-Ig scaffolds or aptamers.

Another subject of the present application is a method for determining the activity of PAM and/or isoforms and/or fragments thereof in a bodily fluid sample of a subject comprising the steps
contacting said sample with a substrate (peptide-Gly) of PAM for an interval of time at t=0 min and t=n+1 min
detecting the reaction product (alpha-amidated peptide) of PAM in said sample at t=0 min and t=n+1 min, and
quantifying the activity of PAM by calculating the difference of the reaction product between t=0 and t=n+1.

Another subject of the present application is a method for determining PAM activity in a bodily fluid sample of a subject comprising the steps
contacting said sample with the substrate ADM-Gly of PAM for an interval of time at t=0 min and t=n+1 min
detecting the reaction product ADM-NH₂ of PAM in said sample at t=0 min and t=n+1 min using an immunoassay, and
quantifying the activity of PAM by calculating the difference of the reaction product ADM-NH₂ between t=0 min and t=n+1 min.

The term “t=n+1 min” is a time interval, wherein n is defined as ≥0 min.

One embodiment of the present application relates to a kit for performing the method for diagnosis or prognosis of a disease in a subject and/or predicting a risk of getting a disease or an adverse event in a subject and/or monitoring a disease or adverse event in a subject, wherein said kit comprises at least two binders directed to recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24).

A specific embodiment of the present application relates to a kit for the detection of the level of PAM comprising one or more binders binding to PAM sequences selected from the group comprising recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24).

Another embodiment of the present application relates to a kit for performing the method for determining the activity of PAM and/or isoforms and/or fragments thereof in a bodily fluid sample of a subject, wherein said kit comprises peptide-Gly PAM as substrate, wherein said peptide-Gly is ADM-Gly.

The activity of PAM can be measured by detection of alpha-amidated peptides (peptide-amide) from their glycinated precursor peptide substrates (peptide-Gly). Nearly half of biologically active peptides terminate with a C-terminal alpha-amide (Vishvanatha et al. 2014. J Biol Chem 289(18):12404-20).

The glycinated precursor peptide substrates may be selected from the group comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin, vasopressin.

In a preferred embodiment said peptide-Gly is adrenomedullin-Gly (ADM-Gly) and said peptide-amide is adrenomedullin-amide (ADM-NH₂).

Other substrates of non-peptide character may comprise N-fatty acyl-glycines, which are converted by PAM to primary fatty acid amides (PFAMs) like oleamide.

In another preferred embodiment of the invention the PAM, e.g., RecPAM, is combined the administration of ascorbate.

Another preferred embodiment of the present application relates to a pharmaceutical formulation, the formulation comprising PAM, ascorbate and/or copper.

Another embodiment of the present application relates to a pharmaceutical formulation, the formulation comprising PAM in combination with ascorbate and/or copper.

In one embodiment, the ascorbic acid compound is L-ascorbic acid or a pharmaceutically acceptable salt thereof; or a pharmaceutically acceptable solvate or hydrate thereof. L-Ascorbic acid is also known as vitamin C, L-xyloascorbic acid, 3-oxo-L-gulofuranolactone (enol form), L-3-ketothreohexuronic acid lactone, antiscorbutic vitamin, cevitamic acid, adenex, allercorb, ascorin, ascorteal, ascorvit, cantan, cantaxin, catavin C, cebicure, cebion, cecon, cegiolan, celaskon, celin, cenetone, cereon, cergona, cescorbat, cetamid, cetabe, cetemican, cevalin, cevatine, cevex, cevimin, ce-vi-sol, cevitan, cevitex, cewin, ciamin, cipca, concemin, C-vin, daviamon C, duoscorb, hybrin, laroscorbine, lemascorb, planavit C, proscorbin, redoxon, ribena, scorbacid, scorbu-C, testascorbic, vicelat, vitacee, vitacimin, vitacin, vitascorbol, and xitix.

In one embodiment, the ascorbic acid compound is L-ascorbic acid. In another embodiment, the ascorbic acid compound is a pharmaceutically acceptable salt of L-ascorbic acid, or a pharmaceutically acceptable solvate or hydrate thereof.

Suitable bases for forming a pharmaceutically acceptable salt of L-ascorbic acid include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

In one embodiment, the ascorbic acid compound is an alkali or alkaline earth metal salt of L-ascorbic acid, or a pharmaceutically acceptable solvate or hydrate thereof. In another embodiment, the ascorbic acid compound is sodium, potassium, calcium, or magnesium L-ascorbate, or a pharmaceutically acceptable solvate or hydrate thereof. In yet another embodiment, the ascorbic acid compound is sodium L-ascorbate, or a pharmaceutically acceptable solvate or hydrate thereof. In yet another embodiment, the ascorbic acid compound is sodium L-ascorbate, which is also known as vitamin C sodium, ascorbin, sodascorbate, natrascorb, cenolate, ascorbicin, or cebitate. In yet another embodiment, the ascorbic acid compound is potassium L-ascorbate, or a pharmaceutically acceptable solvate or hydrate thereof. In yet another embodiment, the ascorbic acid compound is calcium L-ascorbate, or a pharmaceutically acceptable solvate or hydrate thereof. In yet another embodiment, the ascorbic acid compound is calcium L-ascorbate. In yet another embodiment, the ascorbic acid compound is magnesium L-ascorbate, or a pharmaceutically acceptable solvate or hydrate thereof. In still another embodiment, the ascorbic acid compound is magnesium L-ascorbate.

In certain embodiments, the ascorbic acid compound is D-ascorbic acid or a pharmaceutically acceptable salt, or a pharmaceutically acceptable solvate or hydrate thereof.

In one embodiment, the ascorbic acid compound is D-ascorbic acid. In another embodiment, the ascorbic acid compound is a pharmaceutically acceptable salt of D-ascorbic acid, or a pharmaceutically acceptable solvate or hydrate thereof.

Suitable bases for forming a pharmaceutically acceptable salt of D-ascorbic acid include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

In one embodiment, the ascorbic acid compound is an alkali or alkaline earth metal salt of D-ascorbic acid, or a pharmaceutically acceptable solvate or hydrate thereof. In another embodiment, the ascorbic acid compound is sodium, potassium, calcium, or magnesium D-ascorbate, or a pharmaceutically acceptable solvate or hydrate thereof. In yet another embodiment, the ascorbic acid compound is sodium D-ascorbate, or a pharmaceutically acceptable solvate or hydrate thereof. In yet another embodiment, the ascorbic acid compound is sodium D-ascorbate, which is also known as vitamin C sodium, ascorbin, sodascorbate, natrascorb, cenolate, ascorbicin, or cebitate. In yet another embodiment, the ascorbic acid compound is potassium D-ascorbate, or a pharmaceutically acceptable solvate or hydrate thereof. In yet another embodiment, the ascorbic acid compound is calcium D-ascorbate, or a pharmaceutically acceptable solvate or hydrate thereof. In yet another embodiment, the ascorbic acid compound is calcium D-ascorbate. In yet another embodiment, the ascorbic acid compound is magnesium D-ascorbate, or a pharmaceutically acceptable solvate or hydrate thereof. In still another embodiment, the ascorbic acid compound is magnesium D-ascorbate.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a non-crystalline form. Where the solvent is water, the solvate is a hydrate. Examples of hydrates include, but are not limited to, a hemihydrate, monohydrate, dihydrate, trihydrate, tetrahydrate, and pentahydrate.

In one embodiment, the ascorbic acid compound in each of the pharmaceutical compositions is independently L-ascorbic acid or a pharmaceutically acceptable salt thereof, or a pharmaceutically acceptable solvate or hydrate thereof. In another embodiment, the ascorbic acid compound in each of the pharmaceutical compositions is independently an alkali or alkaline earth metal salt of L-ascorbic acid, or a pharmaceutically acceptable solvate or hydrate thereof; or a mixture thereof. In yet another embodiment, the ascorbic acid compound in each of the pharmaceutical compositions is independently sodium, potassium, calcium, or magnesium salt of L-ascorbic acid, or a pharmaceutically acceptable solvate or hydrate thereof; or a mixture thereof. In yet another embodiment, the ascorbic acid compound in each of the pharmaceutical compositions is independently sodium L-ascorbate. In yet another embodiment, the ascorbic acid compound in each of the pharmaceutical compositions is independently calcium L-ascorbate. In yet another embodiment, the ascorbic acid compound in each of the pharmaceutical compositions is independently magnesium L-ascorbate. In still another embodiment, the ascorbic acid compound in each of the pharmaceutical compositions is independently a mixture of two or three of sodium L-ascorbate, calcium L-ascorbate, and magnesium L-ascorbate.

With the above context, the following consecutively numbered embodiments provide further specific aspects of the invention:
1. Peptidylglycine alpha-amidating monooxygenase (PAM) for use as a medicament.
2. PAM for use as a medicament for treatment of a subject, wherein said treatment comprises:
   i. reducing the potential or risk for a disease or disorder, and/or
   ii. reducing the occurrence of a disease or disorder, and/or
   iii. reducing the severity of a disease or disorder.
3. PAM for use as a medicament for treatment of a subject according to embodiment 2, wherein said disease or disorder is selected from the group comprising dementia, cardiovascular disorders, kidney diseases, cancer, inflammatory or infectious diseases and/or metabolic diseases.
4. PAM for use as a medicament for treatment of a subject according to embodiment 2 and 3, wherein said subject is characterized by
   a level of PAM and/or its isoforms and/or fragments thereof below a threshold and/or
   a peptide-Gly/peptide-amide ratio above a threshold
in a sample of bodily fluid of said subject.
5. PAM for use as a medicament for treatment of a subject according to embodiment 4, wherein said peptide is selected from the group of comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin, vasopressin.
6. PAM for use as a medicament for treatment of a subject according to embodiment 4 and 5, wherein said subject is characterized by
   an ADM-Gly/bio-ADM ratio above a threshold and/or
   a bio-ADM concentration below a threshold
in a bodily fluid of said patient.
7. PAM for use as a medicament for treatment of a subject according to embodiment 3, wherein the level of PAM and/or its isoforms and/or fragments thereof is the total concentration of PAM and/or its isoforms and/or fragments thereof having at least 12 amino acids or the activity of PAM and/or its isoforms and/or fragments thereof.
8. PAM for use as a medicament for treatment of a subject according to embodiment 7, wherein the total concentration of PAM and/or its isoforms and/or fragments thereof having at least 12 amino acids or the activity of PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.
9. PAM for use as a medicament for treatment of a subject according to embodiments 4-8, wherein the sample of bodily fluid of said subject is selected from the group of blood, serum, plasma, urine, cerebrospinal fluid (CSF), and saliva.
10. PAM for use as a medicament according to embodiment 1-9, wherein said PAM is selected from the group comprising isolated and/or recombinant and/or chimeric PAM.
11. PAM for use as a medicament according to embodiments 1-10, wherein said recombinant PAM is selected from the sequences comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.
12. PAM for use as a medicament for treatment of a subject according to any embodiment 1 to 11 wherein PAM is combined with ascorbate and/or copper.
13. Pharmaceutical formulation comprising peptidylglycine alpha-amidating monooxygenase (PAM).
14. Pharmaceutical formulation comprising PAM according to embodiment 13, wherein said pharmaceutical formulation is administered orally, epicutaneously, subcutaneously, intradermally, sublingually, intramuscularly, intraarterially, intravenously, or via the central nervous system (CNS, intracerebrally, intracerebroventricularly, intrathecally) or via intraperitoneal administration.
15. Pharmaceutical formulation according to embodiments 13-14, wherein said pharmaceutical formulation is a solution, preferably a ready-to-use solution.
16. Pharmaceutical formulation according to embodiments 13-15, wherein said pharmaceutical formulation is in a freeze-dried state.
17. Pharmaceutical formulation according to embodiments 13-16, wherein said pharmaceutical formulation is administered intra-muscular.
18. Pharmaceutical formulation according to embodiments 13-17, wherein said pharmaceutical formulation is administered intra-vascular.
19. Pharmaceutical formulation according to embodiment 13-18, wherein said pharmaceutical formulation is administered via infusion.
20. Pharmaceutical formulation according to embodiments 13-19, wherein said pharmaceutical formulation is to be administered systemically.
21. Pharmaceutical formulation according to embodiments 13-20, the formulation comprising PAM and/or optionally one or more pharmaceutically acceptable ingredients.
22. Pharmaceutical formulation according to embodiments 13-21, the formulation comprising PAM, ascorbate and/or copper.
23. Pharmaceutical formulation according to embodiments 13-22, the formulation comprising PAM in combination with ascorbate and/or copper.

### FIGURE DESCRIPTION

FIG. 1: Schematic representation of PAM Isoform 1. Black bold arrows indicate cleavage-sites at double-basic amino-acids.

FIG. 2: Enzymatic reaction catalysed by PAM

FIG. 3: Representative calibration curve of recombinant PAM (ADM maturation activity [AMA].

FIG. 4: Frequency distribution (histogram) of AMA in self-reported healthy individuals (n=120)

FIG. 5: Correlation of AMA in matrix duplets (Li-heparin and serum) from self-reported healthy individuals (n=20)

FIG. 6 A-L: Typical calibration curves of PAM sandwich immunoassays. A-J with recombinant PAM as calibration material. (A) solid phase: antibody directed to peptide 10 (SEQ ID No. 20), tracer: antibody directed to peptide 9 (SEQ ID No. 19); (B) solid phase: antibody directed to peptide 10 (SEQ ID No. 20), tracer: antibody directed to peptide 10 (SEQ ID No. 20); (C) solid phase: antibody directed to peptide 9 (SEQ ID No. 19), tracer: antibody directed to peptide 10 (SEQ ID No. 20); (D) solid phase: antibody directed to recombinant PAM (SEQ ID No. 10), tracer: antibody directed to recombinant PAM (SEQ ID No. 10); (E) solid phase: antibody directed to peptide 10 (SEQ ID No. 20), tracer: antibody directed to recombinant PAM (SEQ ID No. 10); (F) solid phase: antibody directed to peptide 13 (SEQ ID No. 23), tracer: antibody directed to peptide 10 (SEQ ID No. 20); (G) solid phase: antibody directed to peptide 14 (SEQ ID No. 24), tracer: antibody directed to peptide 13 (SEQ ID No. 23); (H) solid phase: antibody directed to recombinant PAM (SEQ ID No. 10), tracer: antibody directed to peptide 13 (SEQ ID No. 23); (I) solid phase: antibody directed to peptide 13 (SEQ ID No. 23), tracer: antibody directed to peptide 9 (SEQ ID No. 19); (J) solid phase: antibody directed to peptide 10 (SEQ ID No. 20), tracer: antibody directed to peptide 13 (SEQ ID No. 23). K and L with native PAM (EDTA-Plasma) as calibration material: (K) solid phase: antibody directed to peptide 14 (SEQ ID No. 24), tracer: antibody directed to peptide 13 (SEQ ID No. 23); (L) solid phase: antibody directed to peptide 10 (SEQ ID No. 20), tracer: antibody directed to peptide 13 (SEQ ID No. 23).

FIG. 6 M-O: Enzyme capture assay (ECA)—(M) solid phase antibody directed to peptide 10 (SEQ ID No. 20); (N) solid phase antibody directed to full-length PAM (SEQ ID No. 10); (O) solid phase antibodies directed against peptide 7 (SEQ ID No. 17), peptide 8 (SEQ ID No. 18), peptide 9 (SEQ ID No. 19), peptide 13 (SEQ ID No. 23) and peptide 14 (SEQ ID No. 24) with recombinant PAM/heparin plasma used as sample.

FIG. 7: Typical ADM-Gly dose/signal curve

FIG. 8: ADM maturation activity (PAM activity) in MPP-study (prediction of Alzheimer's disease)

FIG. 9: Kaplan-Meier-Plot (prediction of Alzheimer's disease [AD] in MPP-study)

FIG. 10: ADM maturation activity (PAM activity) in MPP-study (prediction of colorectal cancer [CRC])

FIG. 11: MR-proADM in MPP-study (prediction of colorectal cancer [CRC])

FIG. 12: Kaplan-Meier-Plot (prediction of colorectal cancer [CRC] in MPP-study)

FIG. 13: Kaplan-Meier-Plot (prediction of heart failure in MPP-study)

FIG. 14: Kaplan-Meier-Plot (prediction of atrial fibrillation in MPP-study)

FIG. 15: ADM maturation activity (PAM activity) for diagnosis of prevalent Alzheimer's disease (AD)

FIG. 16: Formation of bio-ADM by native plasma PAM with and without exogenous ADM-Gly as substrate

FIG. 17: Formation of bio-ADM from native plasma ADM-Gly by native plasma PAM and effect of exogenous recombinant PAM

FIG. 18: Shift of ADM-Gly/bio-ADM ratio due to the reaction of native plasma PAM and the effect of exogenous recombinant PAM on the ADM-Gly/bio-ADM ratio

FIG. 19: ADM maturation activity (PAM activity) in rat-plasma prior to and after application of recombinant human PAM or placebo

FIG. 20: One-phase decay fit of ADM maturation activity (PAM activity) in rat plasma after application of recombinant human PAM

FIG. 21 Intravenous injections of placebo (open circles), PAM (closed squares), ascorbate (open triangles) and a combination of both (open squares) in rats. The effect of injected compounds was tested in vitro: (A) PAM-AMA assayed as described in example 3 in absence of exogenous ascorbate. (B) Effect on circulating bio-ADM levels after injection of the compounds. Bio-ADM levels were normalized to levels of Placebo (set as 100%) for each time-point. Significance was tested in comparison to placebo using the two-way ANOVA model with Dunnet's correction. *: p=0.042; **: p=0.0036-0.0073; ***: p=0.0003; ****: p=0.0001; n.s.: not significant.

FIG. 22: Half-life of recombinant PAM in rats, determined from one-phase decay model.

FIG. 23: Amidating activity in healthy human volunteers before (0 h) and after oral ascorbate uptake measured without addition of exogenous ascorbate. AMA at t=0 h was set as 100%.

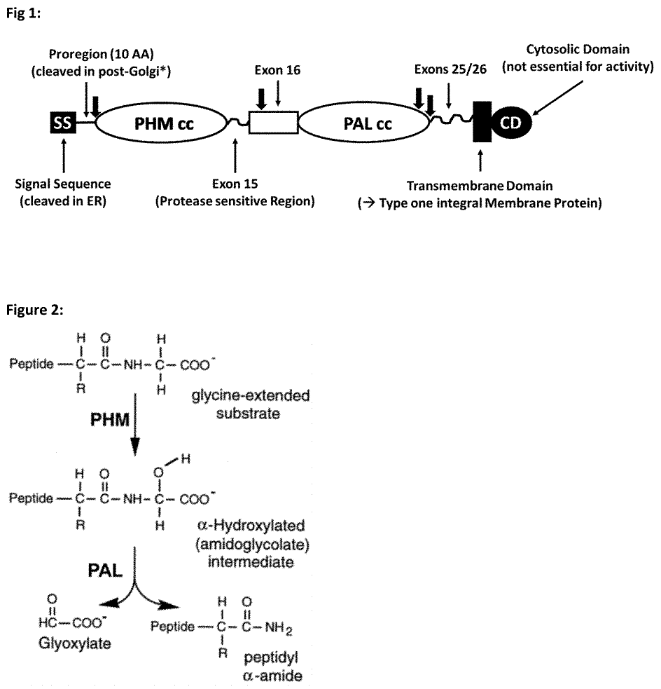

### EXAMPLES

### Example 1—Production of Recombinant PAM

PAM cDNA was synthesized according to Uniprot Accession No. P19021 encoding amino acids 21-834 of the PAM protein involving codon optimization for expression in mammalian cells. The signal sequence of PAM was replaced with human serum albumin signal sequence (MKWVTFISLLFLFSSAYSFR [SEQ ID No. 9]). At the C-terminus of PAM a hexa-histidine tag was added linked via a GS linker to PAM. The sequence of recombinant PAM (amino acids 21-834 of PAM without signal sequence and hexa-histidine tag) is shown in SEQ ID No. 10. The cDNA was cloned into an expression vector (plasmid DNA) using a 5′-NotI and a 3′ HindIII restriction site. The expression vector harboring the cDNA for PAM expression was replicated in- and prepared from E. coli. as a low-endotoxin preparation.

HEK-INV cells were transfected with the expression vector using INVect transfection reagents in serum free suspension culture. The transfection rate was controlled via co-transfection with a GFP—(green fluorescent protein) containing expression vector. Cultivation of cells was carried out in presence of valproic acid and Penicillin-Streptomycin at 37° C. and 5% CO₂. Cells were harvested via centrifugation when viability reached <60% (>2000 g, 30-45 min, 2-8° C.). Cell culture supernatant (CCS) was washed 5 times with 100 mM Tris/HCl, pH 8.0 via tangential flow filtration (TFF, 30 kDa cut-off).

Purification of recombinant PAM included application of buffer exchanged CCS on a Q-sepharose fast flow resin (GE Healthcare) with a NaCl gradient (up to 2 M) elution. Amidating activity containing fractions were pooled and applied onto a Superdex 200 pg (GE Healthcare) size exclusion chromatography column with a 100 mM Tris/HCl, 200 mM NaCl, pH8.0 elution buffer. Amidating activity containing fractions were pooled, dialyzed against 100 mM Tris HCl, 200 mM NaCl, pH 8.0, sterile filtered (0.2 μm). Endotoxin load was determined by Charles River PTS Endosafe system and was below 5 EU/mL.

### Example 2—Production of Antibodies

Anti-PAM antibodies according to the present invention may be synthesised as follows:

PAM peptides for immunization were synthesized, see Table 1, (Peptides & Elephants, Hennigsdorf, Germany) with an additional C-terminal cysteine (if no cysteine is present within the selected PAM-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio. Recombinant PAM was produced by InVivo Biotech Services, Hennigsdorf, as described in example 1.

TABLE 1
PAM immunization peptides

| Name (amino acid position*) | Sequence |
|---|---|
| Peptide 1 (aa 42-56) (SEQ ID No. 11) | CLGTTRPVVPIDSSD |
| Peptide 2 (aa 109-128) (SEQ ID No. 12) | CNMPSSTGSYWFCDEGTCTD |
| Peptide 3 (aa 168-180) (SEQ ID No. 13) | YGDISAFRDNNKD |
| Peptide 4 (aa 204-216) (SEQ ID No. 14) | SVDTVIPAGEKVV |
| Peptide 5 (aa 329-342) (SEQ ID No. 15) | CTQNVAPDMFRTIP |
| Peptide 6 (aa 291-310) (SEQ ID No. 16) | TGEGRTEATHIGGTSSDEMC |
| Peptide 7 (aa 234-244) (SEQ ID No. 17) | YRVHTHHLGKV |
| Peptide 8 (aa 261-276) (SEQ ID No. 18) | QSPQLPQAFYPVGHPV |
| Peptide 9 (aa 530-557) (SEQ ID No. 19) | RGDHVWDGNSFDSKFVYQQIGLGPIEED |
| Peptide 10 (aa 611-631) (SEQ ID No. 20) | EGPVLILGRSMQPGSDQNHFC |
| Peptide 11 (aa 562-579 (SEQ ID No. 21) | IDPNNAAVLQSSGKNLFY |
| Peptide 12 (aa 745-758) (SEQ ID No. 22) | NGKPHFGDQEPVQG |
| Peptide 13 (aa 669-687) (SEQ ID No. 23) | WGEESSGSSPLPGQFTVPH |
| Peptide 14 (aa 710-725) (SEQ ID No. 24) | CFKTDTKEFVREIKHS |
| Recombinant PAM | SEQ ID No. 10 |

*according to SEQ ID No. 1; amino acid (aa)

Balb/c mice were intraperitoneally (i.p.) injected with 100 μg recombinant PAM or 100 μg PAM-peptide-BSA-conjugates at day 0 (emulsified in TiterMax Gold Adjuvant), 100 μg and 100 μg at day 14 (emulsified in complete Freund's adjuvant) and 50 μg and 50 μg at day 21 and 28 (in incomplete Freund's adjuvant). The animal received an intravenous (i.v.) injection of 50 μg recombinant PAM at day 40 or 50 μg PAM-peptide-BSA-conjugates dissolved in saline at day 45. Three days later the mice were sacrificed and the immune cell fusion was performed.

Splenocytes from the immunized mice and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI 1640 culture medium supplemented with 20% fetal calf serum and HAT-Supplement). After one week, the HAT medium was replaced with HT Medium for three passages followed by returning to the normal cell culture medium.

The cell culture supernatants were primarily screened for recombinant PAM binding IgG antibodies two weeks after fusion. Therefore, recombinant PAM (SEQ ID No. 10) was immobilized in 96-well plates (100 ng/well) and incubated with 50 μl cell culture supernatant per well for 2 hours at room temperature. After washing of the plate, 50 μl/well POD-rabbit anti mouse IgG was added and incubated for 1 h at RT.

After a next washing step, 50 μl of a chromogen solution (3.7 mM o-phenylene-diamine in citrate/hydrogen phosphate buffer, 0.012% H₂O₂) were added to each well, incubated for 15 minutes at RT and the chromogenic reaction stopped by the addition of 50 μl 4N sulfuric acid. Absorption was detected at 490 mm.

The positive tested microcultures were transferred into 24-well plates for propagation. After retesting the selected cultures were cloned and re-cloned using the limiting-dilution technique and the isotypes were determined.

Antibodies raised against recombinant human PAM or PAM-peptides were produced via standard antibody production methods (Marx et al. 1997) and purified via Protein A. The antibody purities were ≥90% based on SDS gel electrophoresis analysis.

### Example 3—PAM Activity Assay

Human serum or Li-Heparin plasma from self-reported healthy volunteers was used as source of human native PAM. Each sample (20 μl) was diluted two-fold in 100 mM Tris-HCl in duplicate. The amidation reaction was initiated by addition of 160 μl of PAM-reaction buffer (100 mM Tris-HCl, pH 7.5, 6.25 μM CuSO₄, 2.5 mM L-ascorbate, 125 μg/mL catalase, 62.5 μM amastatin, 250 μM leupeptin, 36 ng/mL synthetic ADM-Gly and 375 μg/mL NT-ADM antibody). Afterwards, 100 μl of each individual reaction of duplicated samples were combined and transferred into 20 μl of 200 mM EDTA to terminate the amidation reaction and to generate t=0 minutes reaction time-point followed by incubation at 37° C. for 40 minutes. Afterwards the non-terminated reactions were stopped with 10 μl of 200 mM EDTA. To determine the PAM activity, bio-ADM as reaction product was quantified in each sample using the Sphingotest® bio-ADM immunoassay (Weber et al. 2017). The amidation assay was calibrated using a 6-point calibration curve generated with human recombinant PAM of known activity. Samples and calibrators were treated in the same manner. Relative light units (RLU t40 min-t0 min) determined via Sphingotest® bio-ADM immunoassay for each sample were fitted against the RLU (t40 min-t0 min) of the calibrator to determine the PAM activity in the samples. PAM activity is described as “adrenomedullin maturation activity” (AMA) in μg bio-ADM formed per hour and L of sample.

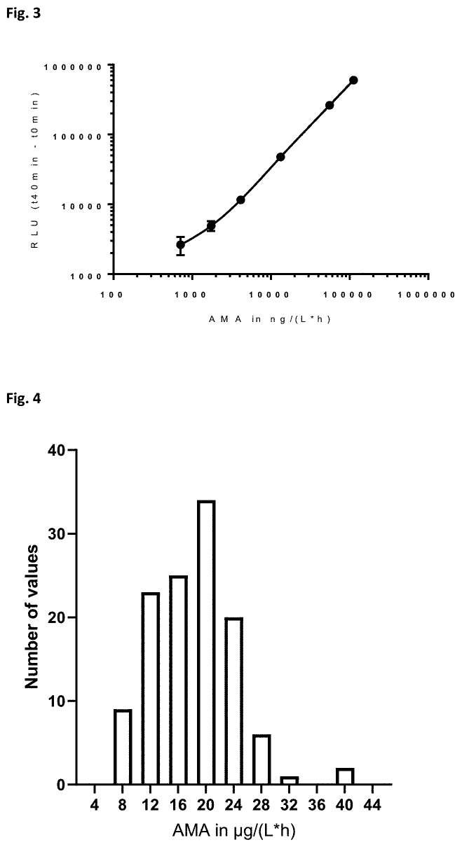

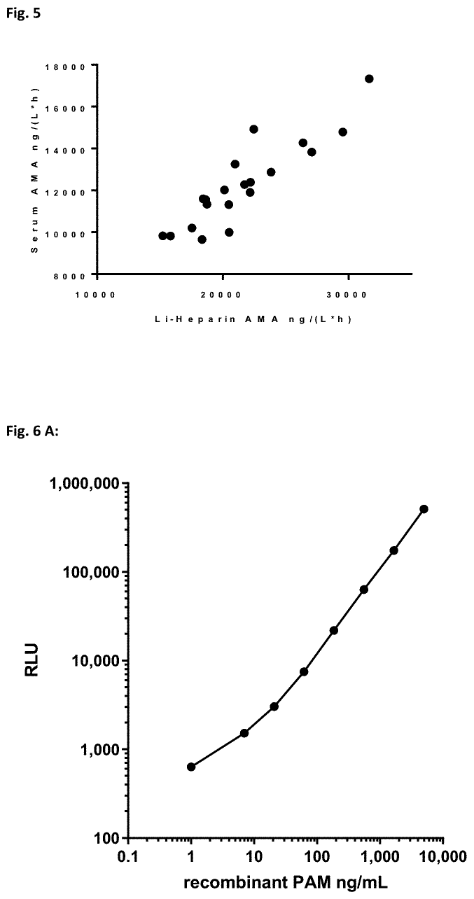

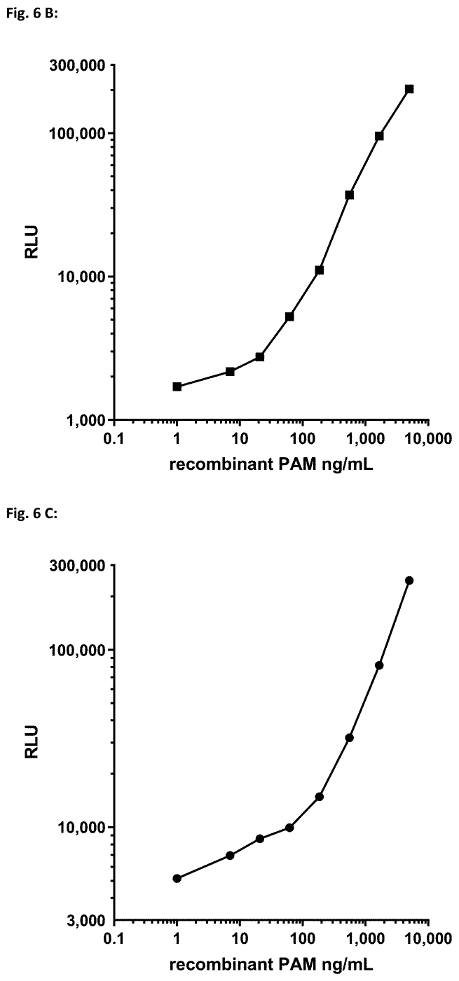

A typical PAM calibration curve is shown in FIG. 3. The distribution of AMA in Li-Heparin samples from n=120 self-reported healthy volunteers are shown in FIG. 4. The median [IQR] of Li-Heparin AMA was 18.4 μg/(L*h) [13.5-21.9]. The 10^{th} and 90^{th} percentile was 10.5 and 24.2 μg/(L*h), respectively. The 2.5^{th}, 97.5^{th} and 99^{th} percentile was 8.1, 31.6 and 40.8 μg/(L*h). In addition, matched serum samples from n=20 subjects were measured and revealed a highly significant correlation (r=0.89; p<0.0001) (FIG. 5), although AMA values in serum were approximately 40% lower when compared to Li-Heparin.

### Example 4—PAM Immunoassays

Antibodies against recombinant PAM (SEQ ID No. 10) and PAM peptides (SEQ ID No. 11 to 24) were raised as described in example 1.

The technology used was a sandwich luminescence immunoassay, based on Akridinium ester labelling.

4.1. Labelled Compound (Tracer)

Purified antibodies (0.2 g/L) were labelled by incubation in 10% labelling buffer (500 mmol/L sodium phosphate, pH 8.0) with 1:5 mol/L ratio of MACN-acridinium-NHS-ester (1 g/L, InVent GmbH) for 20 min at 22° C. After adding 5% 1 mol/L Tris-HCl, pH 8.0, for 10 min, the respective antibody was separated from free label via CentriPure P10 columns (emp Biotech GmbH). The purified labelled antibody was diluted in 300 mmol/l potassium phosphate, 100 mmol/l NaCl, 10 mmol/l Na-EDTA, 5 g/l Bovine Serum Albumin (pH 7.0). The final concentration was approximately 20 ng of labelled antibody per 150 μL.

4.2. Solid Phase

White polystyrene microtiter plates (Greiner Bio-One International AG) were coated (18 h at 20° C.) with the respective antibody (2 μg/0.2 mL per well 50 mmol/L Tris-HCl, 100 mmol/L NaCl, pH 7.8). After blocking with 30 g/L Karion, 5 g/L BSA (protease free), 6.5 mmol/L monopotassium phosphate, 3.5 mmol/L sodium dihydrogen phosphate (pH 6.5), the plates were vacuum-dried.

4.3 Calibration

The assay was calibrated, using dilutions of recombinant PAM as described in Example 1. The typical concentration range was within of 5-5,000 ng/mL.

4.4. PAM Immunoassays:

4.4.1. PAM-LIA

One-Step version: 50 μL of samples/calibrators were pipetted into pre-coated microtiter plates. After adding 200 μL of labelled antibody in buffer (300 mmol/L potassium phosphate, 100 mmol/L NaCl, 10 mmol/L Na-EDTA, 50 μmol/L amastatin, 100 μmol/L leupeptin, 0.1% bovine IgG, 0.02% mouse IgG, 0.5% BSA, pH 7.0), the microtiter plates were incubated for 20 h at 2-8° C. under agitation at 600 rpm. Unbound tracer was removed by washing 5 times (each 350 μL per well) with washing solution (20 mmol/L PBS, 1 g/L Triton X-100, pH 7.4). Wellbound chemiluminescence was measured for 1 s per well by using the Centro LB 960 microtiter plate luminescence reader (Berthold Technologies).

Two-Step version: 50 μL of samples/calibrators were pipetted into pre-coated microtiter plates. After adding 200 μL of buffer (as described in one-step version), the microtiter plates were incubated for 15-20 h at 2-8° C. under agitation at 600 rpm. Unbound sample was removed by washing 4 times (each 350 μL per well) with washing solution with subsequent addition of 200 μl of tracer material and incubation of microtiter plates at room temperature for 2 h. Unbound tracer was removed by washing 4 times (each 350 μL per well) with washing solution. Wellbound chemiluminescence was measured for 1 s per well by using the Centro LB 960 microtiter plate luminescence reader (Berthold Technologies).

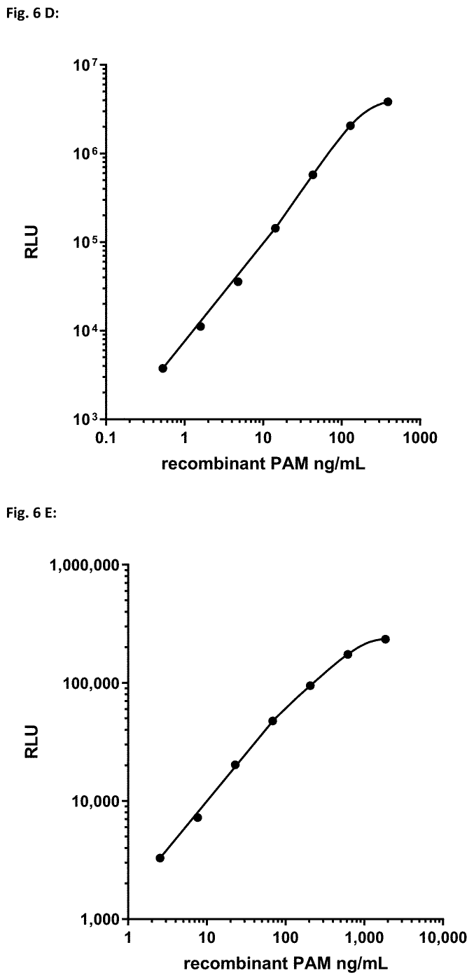

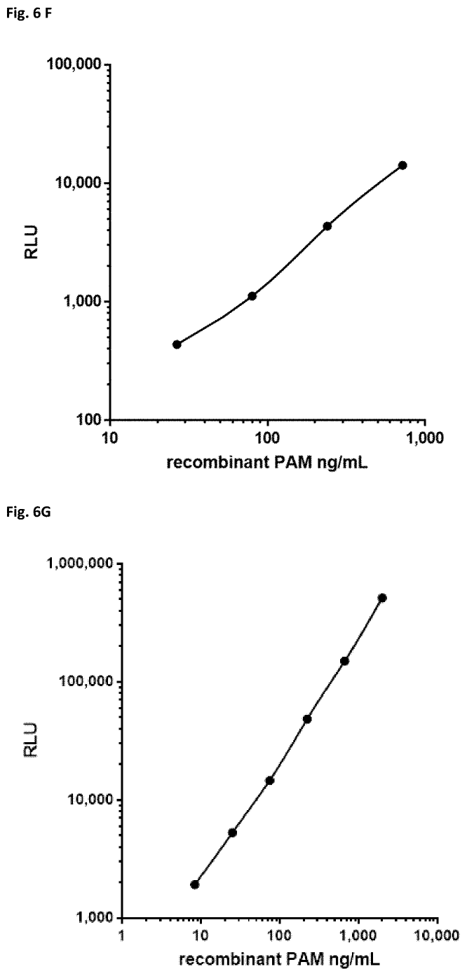

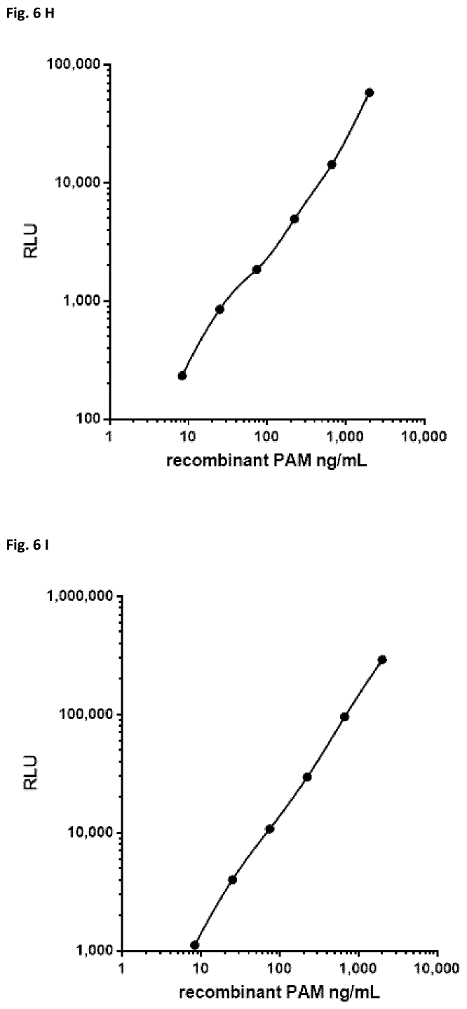

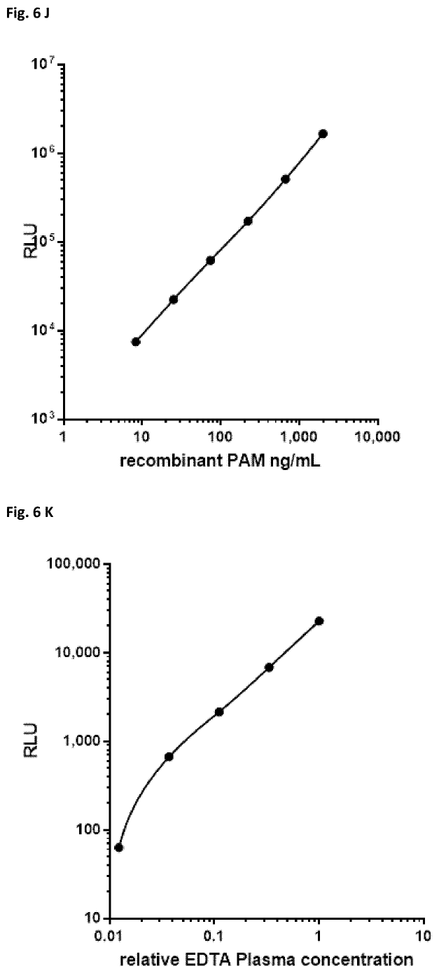

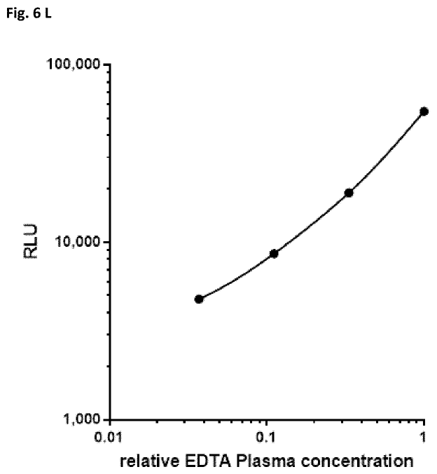

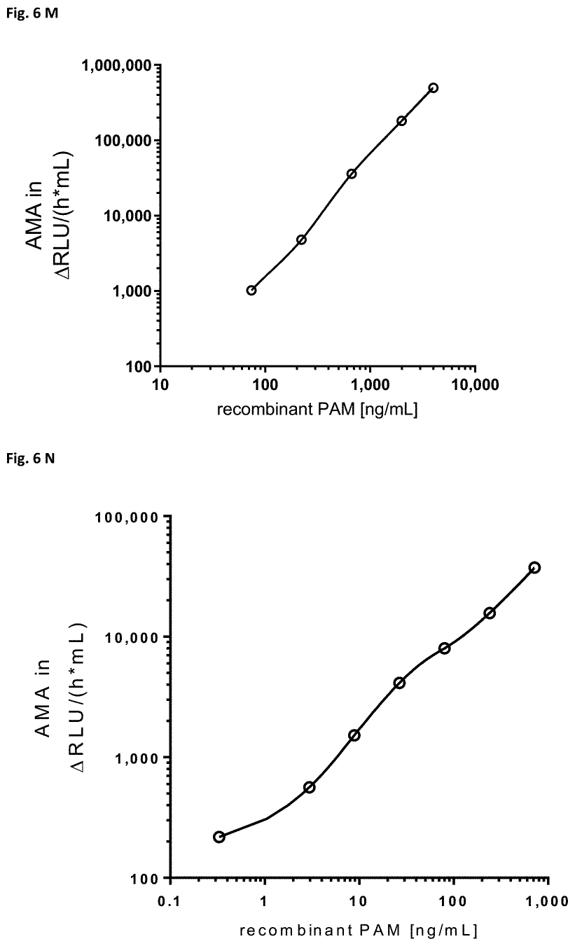

Results: Antibodies bound to the solid phase and labelled antibodies directed to the different PAM immunization peptides as well as full-length (recombinant) PAM (see example 2) were tested with recombinant PAM as well as blood samples. Exemplary standard curves for different antibody combinations are shown in FIG. 6 (A-L). FIGS. 6 (A-J) with recombinant PAM as calibration material: (A) solid phase: antibody directed to peptide 10 (SEQ ID No. 20), tracer: antibody directed to peptide 9 (SEQ ID No. 19); (B) solid phase: antibody directed to peptide 10 (SEQ ID No. 20), tracer: antibody directed to peptide 10 (SEQ ID No. 20); (C) solid phase: antibody directed to peptide 9 (SEQ ID No. 19), tracer: antibody directed to peptide 10 (SEQ ID No. 20); (D) solid phase: antibody directed to recombinant PAM (SEQ ID No. 10), tracer: antibody directed to recombinant PAM (SEQ ID No. 10); (E) solid phase: antibody directed to peptide 10 (SEQ ID No. 20), tracer: antibody directed to recombinant PAM (SEQ ID No. 10); (F) solid phase: antibody directed to peptide 13 (SEQ ID No. 23), tracer: antibody directed to peptide 10 (SEQ ID No. 20); (G) solid phase: antibody directed to peptide 14 (SEQ ID No. 24), tracer: antibody directed to peptide 13 (SEQ ID No. 23); (H) solid phase: antibody directed to recombinant PAM (SEQ ID No. 10), tracer: antibody directed to peptide 13 (SEQ ID No. 23); (I) solid phase: antibody directed to peptide 13 (SEQ ID No. 23), tracer: antibody directed to peptide 9 (SEQ ID No. 19); (J) solid phase: antibody directed to peptide 10 (SEQ ID No. 20), tracer: antibody directed to peptide 13 (SEQ ID No. 23). FIGS. 6 (K and L) with native PAM (EDTA-Plasma) as calibration material: (K) solid phase: antibody directed to peptide 14 (SEQ ID No. 24), tracer: antibody directed to peptide 13 (SEQ ID No. 23); (L) solid phase: antibody directed to peptide 10 (SEQ ID No. 20), tracer: antibody directed to peptide 13 (SEQ ID No. 23). With all antibody combinations PAM was also detectable in human plasma and serum samples.

4.4.2. Enzyme Capture Assay (ECA) for the Detection of PAM Activity

Enzyme capture assays were established to detect the activity of PAM. 50 μL of samples/calibrators were pipetted into pre-coated microtiter plates (as described in 4.2). After adding 200 μL of buffer (300 mmol/L potassium phosphate, 100 mmol/L NaCl, 50 μmol/L amastatin, 100 μmol/L leupeptin, 0.1% bovine IgG, 0.02% mouse IgG, 0.5% BSA, pH 7.0) the microtiter plates were incubated for 1 h at room temperature under agitation at 600 rpm. Unbound sample was removed by washing 4 times (each 350 μL per well) with washing solution with subsequent addition of 200 μl reaction buffer per well and incubation at 37° C. Reaction buffer including all components and final concentrations were as described in Example 3, with the exceptions that 100 pg/mL NT-ADM-antibody and 288 ng/mL ADM-Gly were used. Reaction was terminated at several time-points by transferring 10 μl of each individual reaction into 190 μl of EDTA containing buffer (300 mmol/L potassium phosphate, 100 mmol/L NaCl, 10 mmol/L Na-EDTA, 50 μmol/L amastatin, 100 μmol/L leupeptin, 0.1% bovine IgG, 0.02% mouse IgG, 0.5% BSA, pH 7.0). Terminated reactions were applied onto the Sphingotest® bio-ADM immunoassay for quantification of produced bio-ADM. A typical standard curve using an antibody directed to PAM immunization peptide 10 (SEQ ID No. 20) as solid phase is shown in FIG. 6 M.

FIG. 6 N shows a typical standard curve using an antibody directed to full-length recombinant PAM (SEQ ID No. 10). Further antibodies directed to peptide 7 (SEQ ID No. 17), peptide 8 (SEQ ID No. 18), peptide 9 (SEQ ID No. 19), peptide 13 (SEQ ID No. 23) and peptide 14 (SEQ ID No. 24) were used as solid phase for the enzyme capture assay and a sample (250 μl) of recombinant PAM or heparin plasma was measured for PAM activity (FIG. 6 O). These results show, that the antibodies can be used to detect PAM activity in human samples using the ECA technique. PAM was also detectable in plasma and serum samples.

In a further step, PAM activity (as described in example 3) and PAM concentration using a PAM-LIA (solid phase antibody directed against full-length PAM, tracer antibody directed against peptide 13 [SEQ ID No. 23]) were determined in heparin samples from healthy volunteers (n=26). PAM activity and PAM concentration correlated significantly as shown in FIG. 6 P (Spearman r=0.49, p=0.0109).

### Example 5—ADM-Gly Immunoassay

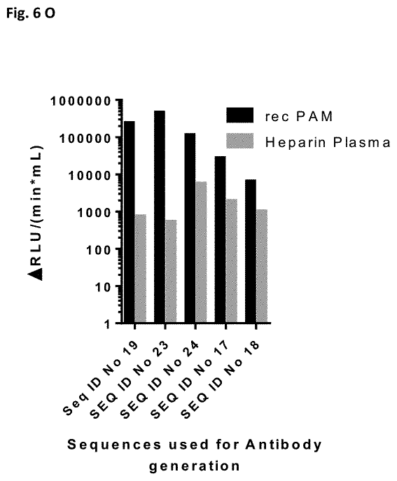

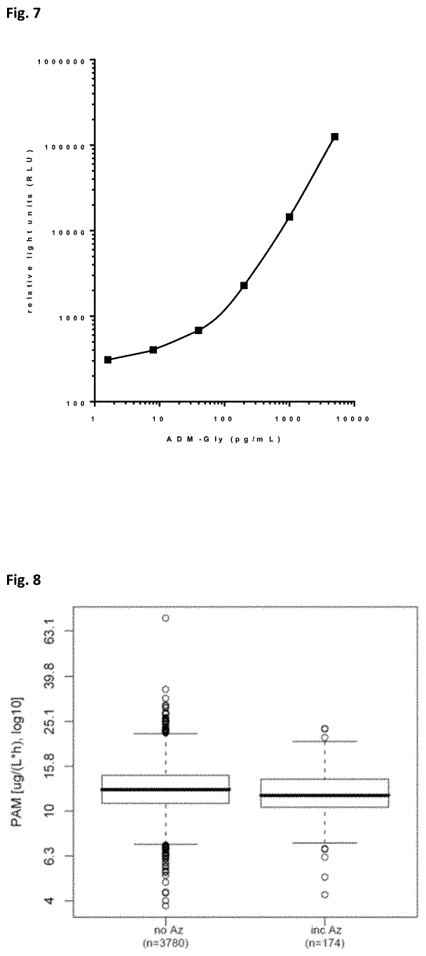

ADM-Gly was quantified as based on Weber et al. (Weber et al. 2017. JALM 2(2): 222-233) for bioactive ADM with the following modifications: the tracer-antibody used for ADM-Gly detection, labelled with MACN-acridinium-NHS, was directed to the C-terminal glycine of ADM-Gly. The assay was calibrated with synthetic ADM-Gly. The limit of detection (LOD) was 10 pg/mL of ADM-Gly. Cross-reactivity of antibody directed to the C-terminal glycine of ADM with bio-ADM was in the range between 6 and 50% in a concentration dependent manner. All determined ADM-Gly concentrations were corrected for cross-reactivity as follows: For each ADM-Gly quantification additional quantification of bio-ADM in corresponding samples was performed using the Sphingotest® bio-ADM immunoassay. The corresponding bio-ADM values were used to determine the signal (RLU) generated with the antibody directed to C-terminal glycine of ADM on a bio-ADM calibration curve. The determined signal (RLU) was used to calculate the false-positive ADM-Gly concentration (pg/mL) using the ADM-Gly calibration curve. This concentration was subtracted from the initially determined ADM-Gly concentration. A typical standard curve is shown in FIG. 7.

### Example 6—Prediction of Diseases in Healthy Subjects

The Malmö Preventive Project (MPP) was funded in the mid-1970s to explore CV risk factors in general population, and enrolled 33,346 individuals living in Malmö (Fedorowski et al. 2010. Eur Heart J 31: 85-91). Between 2002 and 2006, a total of 18,240 original participants responded to the invitation (participation rate, 70.5%) and were screened including a comprehensive physical examination and collection of blood samples (Fava et al. 2013. Hypertension 2013; 61: 319-26). The re-examination in MPP is in the present study regarded as the baseline. Subjects with prior CVD at baseline were excluded. An informed consent was obtained from all participants and the Ethical Committee of Lund University, Lund, Sweden, approved the study protocol.

A commercial fully automated homogeneous time-resolved fluoro-immunoassay was used to measure MR-proADM in plasma (BRAHMS MR-proADM KRYPTOR; BRAHMS GmbH, Hennigsdorf, Germany) (Caruhel et al. 2009. Clin Biochem. 42 (7-8):725-8).

Bio-ADM was measured as described by Weber et al. 2017 (Weber et al. 2017. JAMA 2(2): 222-233). AMA was determined in 4942 serum samples from MPP as described in example 3. Each sample was measured in duplicate. Samples, controls and calibrators were treated in the same manner. Baseline clinical characteristics of AMA after stratification to Quartiles is shown in table 2.

TABLE 2
Baseline clinical characteristics according to quartile
(Q) of AMA at baseline of subjects analysed

| | Q1 | | Q2 | | Q3 | | Q4 | | |
|---|---|---|---|---|---|---|---|---|---|
| | (n = 1235) | | (n = 1236) | | (n = 1236) | | (n = 1235) | | p |
| AMA in μg/(L*h) (SD) | 9.416 | (1.21) | 11.66 | (0.46) | 13.39 | (0.57) | 17 | (3) | N/A |
| AMA range | 3.8-10.86 | | 10.86-12.47 | | 12.47-14.47 | | 14.47-72.15 | | N/A |
| Age in years (SD) | 68.97 | (6.18) | 69.16 | (6.28) | 69.34 | (6.38) | 70.45 | (6.07) | <0.0001 |
| Current smoking, n (%) | 188 | (15.2) | 217 | (17.6) | 255 | (20.6) | 287 | (23.2) | <0.0001 |
| Systolic blood | 144 | (19.77) | 145.1 | (19.83) | 144.8 | (20.33) | 147.6 | (21.34) | <0.0002 |
| pressure in mmHg (SD) | | | | | | | | | |
| Diastolic blood | 82.83 | (10.12) | 84.04 | (10.83) | 83.12 | (10.61) | 84.45 | (11.51) | 0.0041 |
| pressure mmHg (SD) | | | | | | | | | |
| Diabetes Mellitus, n (%) | 166 | (13.4) | 127 | (10.3) | 113 | (9.1) | 127 | (10.3) | 0.0043 |
| Glucose in mmol/L (SD) | 6.024 | (1.95) | 5.78 | (1.21) | 5.794 | (1.37) | 5.753 | (1.28) | 0.0299 |

N/A: not applicable

Statistical analysis: Values are expressed as means and standard deviations, medians and interquartile ranges (IQR), or counts and percentages as appropriate. Group comparisons of continuous variables were performed using the Kruskal-Wallis test. Biomarker data were log-transformed. Cox proportional-hazards regression was used to analyze the effect of risk factors on survival in uni- and multivariable analyses. The assumptions of proportional hazard were tested for all variables. For continuous variables, hazard ratios (HR) were standardized to describe the HR for a biomarker change of one IQR. 95% confidence intervals (CI) for risk factors and significance levels for chi-square (Wald test) are given. The predictive value of each model was assessed by the model likelihood ratio chi-square statistic. The concordance index (C index) is given as an effect measure. It is equivalent to the concept of AUC adopted for binary outcome. For multivariable models, a bootstrap corrected version of the C index is given. Survival curves plotted by the Kaplan-Meier method were used for illustrative purposes. To test for independence of PAM from clinical variables we used the likelihood ratio chi-square test for nested models. All statistical tests were 2-tailed and a two-sided p-value of 0.05 was considered for significance.

6.2. Prediction of Alzheimer's Disease

3954 samples with information about dementia diagnosis were selected (n=174 with incident AD). Information about dementia diagnoses was requested from the Swedish National Patient Register (SNPR). The diagnoses in the register were collected according to different revisions of the International Classification of Diseases (ICD) codes 290, 293 (ICD-8), 290, 331 (ICD-9) or F00, FO1, F03, G30 (ICD-10). Since 1987, SNPR includes all in-patient care in Sweden and, in addition, contains data on outpatient visits including day surgery and psychiatric care from both private and public caregivers recorded after 2000. All-cause dementia was diagnosed according to the criteria of the Diagnostic and Statistical Manual of Mental Disorders (DSM)-III revised edition, whilst the DSM-IV criteria were applied for the Alzheimer's disease and vascular dementia diagnoses. Diagnoses were validated by a thorough review of medical records as well as neuroimaging data when available. A research physician assigned the final diagnosis for each patient and a geriatrician specialized in cognitive disorders was consulted in unresolved cases. The PAM activity (AMA) was determined as described in example 3. AMA in the MPP cohort is shown in FIG. 8: AMA in patients developing AD over time (incident AD, n=174) are significantly lower compared to the non-AD group (p=0.01).

Reduced serum AMA strongly predicts Alzheimer's disease with a Hazard Ratio (HR) of 0.74 (CI 0.6-0.88; p<0.001) and a HR of 0.72 (CI 0.6-0.85) when adjusted for age (table 3). FIG. 9 shows a Kaplan-Meier Plot for the prediction of Alzheimer's disease using AMA (prevalent AD cases were excluded from the analysis). The lowest tertile is associated with the highest risk of getting AD.

Furthermore, AMA as a predictor of AD was independent from bio-ADM concentrations. Both markers contribute to AD prediction. While the C-Index for AMA alone is 0.571 (CI 0.525-0.616; Chi² 10.97) the C-index for both combined markers, i.e., AMA and bio-ADM is 0.595 (Chi² 18.96; p<0.0001). Moreover, AMA in combination with bio-ADM and MR-proADM concentrations further improve the prediction of incident Alzheimer. While MR-proADM alone had no predictive value for AD, the combination of AMA, bio-ADM and MR-proADM showed a C-index of 0.622 (Chi² 26.73; p=0.00001).

TABLE 3
Prediction of Alzheimer's disease

| | Hazard Ratio | | | |
|---|---|---|---|---|
| Biomarker | (HR) (CI) | p-Value | C-Index (CI) | Chi² |
| AMA | 0.72 | p < 0.001 | 0.571 | 10.97 |
| | (0.6-0.85) | | (0.525-0.616) | |
| AMA, | | p < 0.0001 | 0.595 | 18.96 |
| bio-ADM | | | | |

6.3. Prediction of Colorectal Cancer (CRC)

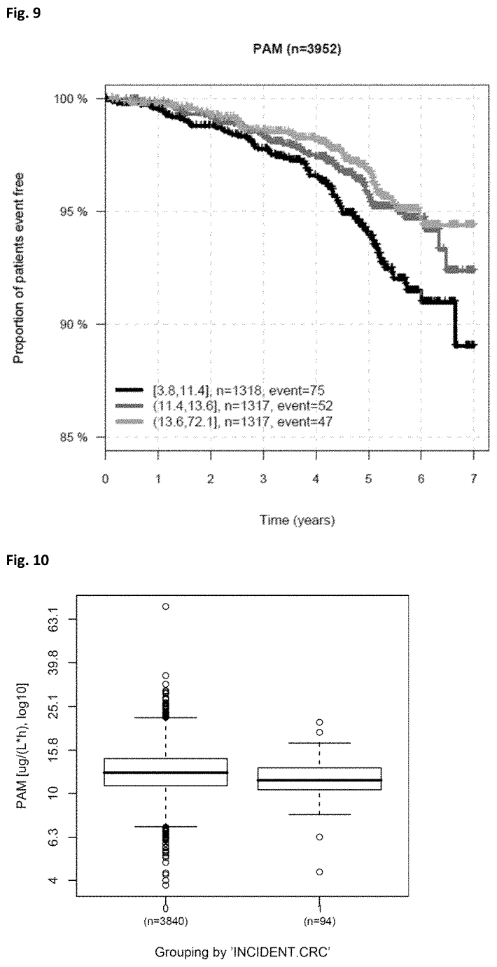

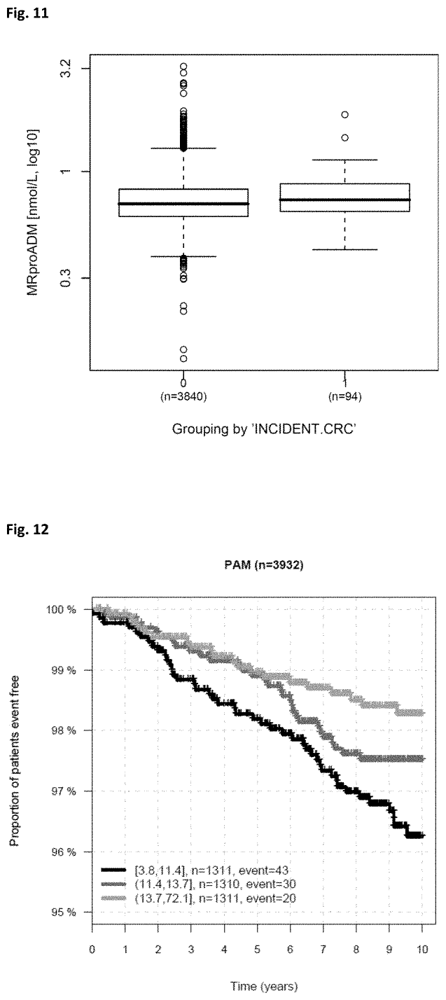

AMA of subjects with and without incident CRC is shown in FIG. 10. The AMA in patients developing CRC over time (n=93) are significantly lower compared to the non-CRC group (p=0.0008; Kruskal-Wallis). In contrast, the MR-proADM concentrations in patients developing CRC over time are higher compared to the non-CRC group (p=0.023) as shown in FIG. 11.

Results for the single markers and marker combinations are shown in Table 4. Reduced serum AMA (age-adjusted) strongly predicts development of CRC with a Hazard Ratio (HR) of 0.68 (p<0.0001). FIG. 12 shows a Kaplan-Meier Plot for the prediction of CRC with AMA (prevalent cases were excluded from the analysis). The lowest tertile is associated with the highest risk of CRC development (p<0.005).

Increased MR-proADM concentrations predict development of CRC with a HR of 1.36 p<0.05). The highest quartile is associated with the highest risk of CRC development (p=0.051).

While bio-ADM concentrations were not predictive for development of CRC, a combination of bio-ADM and AMA showed an improved CRC prediction (see table 4). In addition, a combination of AMA and MR-proADM further improved the prediction of CRC development.

In summary, reduced AMA values predict development of CRC. Increased MR-proADM concentrations also predict development of CRC. A combination of AMA with bio-ADM or MR-proADM enhances the predictive value for CRC.

TABLE 4
Prediction of colorectal cancer

| | Hazard Ratio | | | |
|---|---|---|---|---|
| Biomarker | (HR) (CI) | p-Value | C-Index (CI) | Chi² |
| AMA | 0.68 | p < 0.00001 | 0.588 | 8.51 |
| | (0.6-0.85) | | (0.535-0.641) | |
| AMA, | | p < 0.002 | 0.598 | 12.48 |
| bioADM | | | | |
| MR-proADM | 1.36 | p < 0.05 | 0.587 | 6.27 |
| | (1.08-1.72) | | (0.532-0.642) | |
| AMA, | | p < 0.0005 | 0.612 | 16.51 |
| MR-proADM | | | | |

6.5. Prediction of Cardiovascular Disorders

Cardiovascular disorder analyses were performed in 4942 samples with information about death- and cardiovascular events from the MPP cohort. Information about cardiovascular events and diagnoses was requested from the Swedish National Patient Register (SNPR). The diagnoses in the register were collected according to different revisions of the International Classification of Diseases (ICD) codes.

Since 1987, SNPR includes all in-patient care in Sweden and, in addition, contains data on outpatient visits including day surgery and psychiatric care from both private and public caregivers recorded after 2000. The PAM activity (AMA) was determined as described in example 3. Within of the total set of 4942 serum samples from the MPP study cohort 278 subjects developed heart failure (incident heart failure) and 633 subjects developed atrial fibrillation (incident atrial fibrillation) during follow-up period of 12.8 years.

Elevated serum AMA strongly predicts incident heart failure (83 prevalent HF cases were excluded from the analyses) with a Hazard Ratio (HR) of 1.537 (CI 1.169-2.021; p<0.0007) (Table 5). FIG. 13 shows a Kaplan-Meier Plot for the prediction of heart failure using AMA. High AMA is associated with increased risk of getting heart failure.

Elevated serum AMA strongly predicts incident atrial fibrillation (267 prevalent AF cases were excluded from the analyses) with a Hazard Ratio (HR) of 1.459 (CI 1.214-1.752; p<0.0001) (Table 5). FIG. 14 shows a Kaplan-Meier Plot for the prediction of atrial fibrillation using AMA. High AMA is associated with increased risk of getting atrial fibrillation.

TABLE 5
Prediction of cardiovascular disorders
Heart failure

| | Q1 | Q2 |
|---|---|---|
| | (n = 3707) | (n = 1119) |
| Number of Events | 186 | 92 |
| Logrank Hazard | (ref) | 1.537 (1.169-2.021) |
| Ratio (95% CI) | | |
| Chi² | | 11.56 |
| p-value | | =0.0007 |

Atrial fibrillation

| | Q1 | Q2 |
|---|---|---|
| | (n = 3534) | (n = 1141) |
| Number of Events | 436 | 197 |
| Logrank Hazard | (ref) | 1.459 (1.214-1.752) |
| Ratio (95% CI) | | |
| Chi² | | 19.59 |
| p-value | | <0.0001 |

### Example 7—Diagnosis of Diseases

7.1. Diagnosis of Alzheimer's Disease

Serum samples from 27 individuals with diagnosed Alzheimer's disease were obtained from InVent Diagnostica GmbH. The AD diagnosis is based on cognitive tests (CERAD, DemTec, MMST and Clock-Drawing test) as well as on MRI (Magnetic resonance imaging) and CT-scans. As controls, 67 serum samples from self-reported healthy volunteers were used. AMA was detected as described in example 3.

As shown in FIG. 15 patients from the AD-cohort showed significantly lower serum AMA when compared to the control-cohort (n=67; p<0.0001).

7.2. Diagnosis of Cardiovascular and Metabolic Disorders

In the total set of 4942 serum samples from the MPP study cohort, 267 cases of prevalent atrial fibrillation, 83 cases of prevalent chronic heart failure and 533 cases of prevalent diabetes were present. Significant elevation of serum AMA (p<0.0001) was observed in prevalent atrial fibrillation (mean AMA: 13.92 AMA-Units, n=267) when compared to individuals free of prevalent atrial fibrillation (mean AMA: 12.8 AMA-Units, n=4675). Significant elevation of serum AMA (p=0.0019) was observed in prevalent chronic heart failure (mean AMA: 14.31 AMA-Units, n=83) when compared to individuals free of prevalent heart failure (mean AMA: 12.84 AMA-Units, n=4859). Significant reduction of serum AMA (p=0.0035) was observed in prevalent diabetes (mean AMA: 12.69 AMA-Units, n=533) when compared to individuals free of prevalent diabetes (mean AMA: 12.89 AMA-Units, n=4409).

### Example 8—Conversion of ADM-Gly to Bio-ADM by Native and Recombinant PAM

a) Conversion of ADM-Gly to Bio-ADM by Native PAM

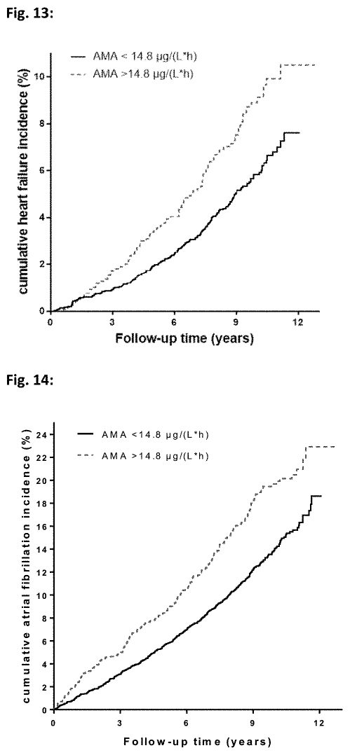

Human Li-Heparin plasma (pool of 3 specimen with low ADM-Gly (<50 pg/mL)) was used as source of human native PAM. The amidation reaction was performed in a total volume of 120 μl at 37° C. 96 μl of plasma were spiked with ADM-Gly (5 ng/mL final concentration). As control, equal volumes of 100 mM Tris-HCl, pH 7.5 were added to untreated plasma. The prepared samples were allowed to chill for 15 minutes at room temperature. The amidation reaction was started by addition of 24 μl of PAM-reaction buffer resulting in final concentrations of 2 mM L-ascorbate and 5 μM CuSO4, respectively, with a final concentration for ADM-Gly 4 ng/mL. After 0 min, 30 min, 60 min and 90 min of incubation at 37° C., the reaction was stopped by addition of Na-EDTA (20 mM final concentration). The concentration of bio-ADM in the reaction sample was quantified using the Sphingotest® bio-ADM immunoassay as described recently (Weber et al. 2017. JALM 2(2): 222-233). No change in bio-ADM concentration was detected in low ADM-Gly samples without addition of exogenous ADM-Gly. When ADM-Gly was added to the sample, a linear formation of bio-ADM was detected within 90 minutes (FIG. 16).

b) Conversion of ADM-Gly to Bio-ADM by Exogenous (Recombinant) PAM

In a further experiment we investigated the formation of bio-ADM from endogenous ADM-Gly by native human plasma PAM and the effect of addition of exogenous recombinant human PAM. Human Li-Heparin plasma (pool of 5 specimen with high ADM-Gly (>400 pg/mL)) was used as source of human native PAM and human native ADM-Gly. The amidation reaction was performed in a total volume of 315 μl at 37° C. 250 μl of plasma were spiked with 65 μl reaction buffer with or without recombinant PAM (see Example 1) to initiate the amidation reaction. Final concentrations in the reaction samples were: 2 mM L-ascorbate, 5 μM CuSO4, 50 μM Amastatin, 200 μM Leupeptin and 100 μg/mL Catalase. The concentration of exogenous recombinant PAM was 500 ng/ml. After 0 min, 30 min, 55 min and 80 min of incubation at 37° C., the reaction was stopped by addition of Na-EDTA (20 mM final concentration). The concentration of bio-ADM in the reaction sample was quantified using the Sphingotest® bio-ADM immunoassay as described recently (Weber et al. 2017. JALM 2(2): 222-233). The concentrations of ADM-Gly were determined as described in example 5.

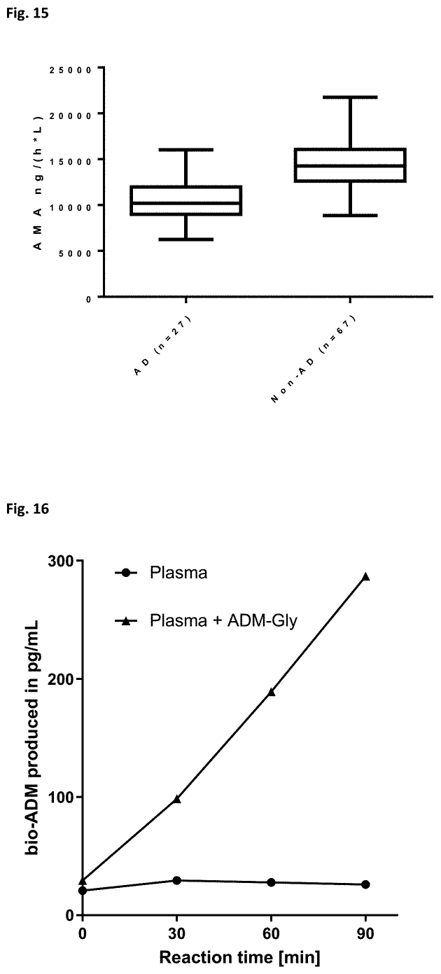

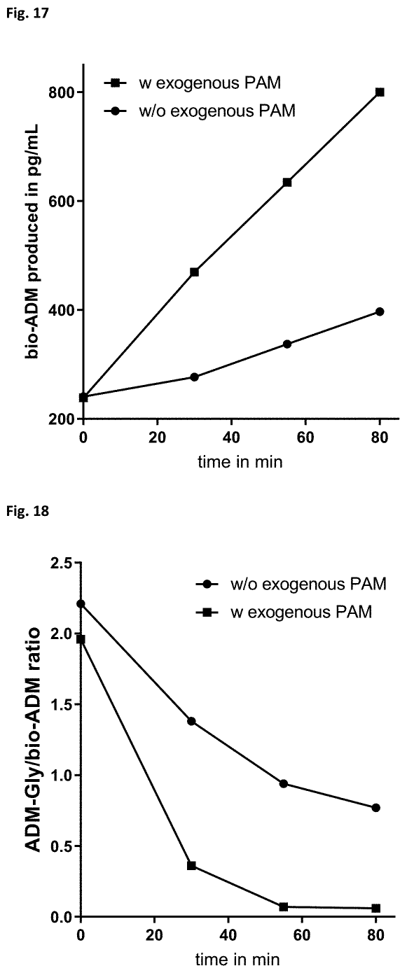

As shown in FIG. 17, endogenous human PAM enzyme is capable of the conversion of endogenous human ADM-Gly to bioADM in a time-dependent manner. Moreover, ADM-Gly/bio-ADM ratio is shifted towards bio-ADM in a time dependent manner (FIG. 18). These data clearly indicate that the PAM exerts its function of c-terminal amidation of peptide-hormones not only in the lumen of secretory vesicles but also in the circulation. An approximate doubling of the PAM concentration in the reaction sample by addition of exogenous recombinant human PAM leads to an increase of the bio-ADM synthesis rate from endogenous human ADM-Gly (FIG. 17) by an averaged factor of 1.8 at each time-point. Furthermore, the ADM-Gly consumption is increased with a faster shift of the ADM-Gly/bio-ADM ratio towards bio-ADM (FIG. 18). These in vitro findings show an unexpectedly high potential of recombinant human PAM in shifting potentially unfavourable circulating ADM-Gly/bio-ADM ratios towards bio-ADM.

### Example 9—Application of Recombinant PAM (In Vivo Half-Life of PAM in Rats)

Two animals (Wistar-rat, male, 2-3 month of age) received 17 μg of recombinant human PAM (see example 1) as a single dose in a total volume of 500 μl (in phosphate-buffered saline). One control-animal received 500 μl of PBS. Blood sampling (Li-Heparin Plasma) was carried out 30 min prior to application and 30 min, 2 h, 4 h, 8 h, 24 h and 48 h, respectively, after application.

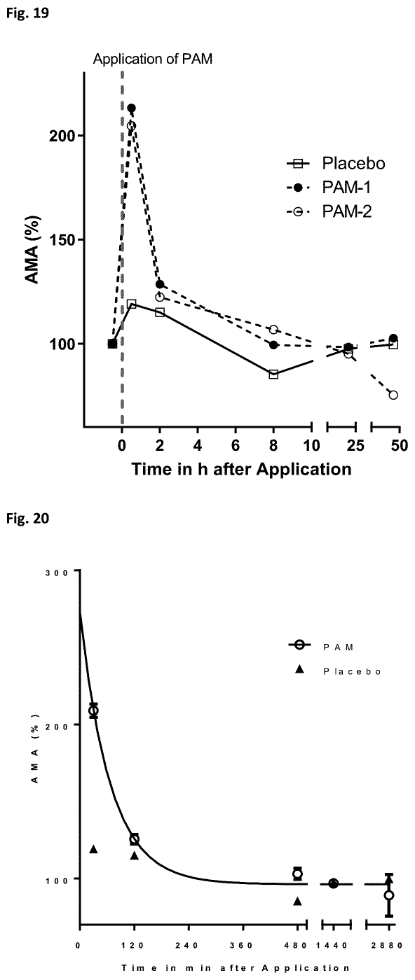

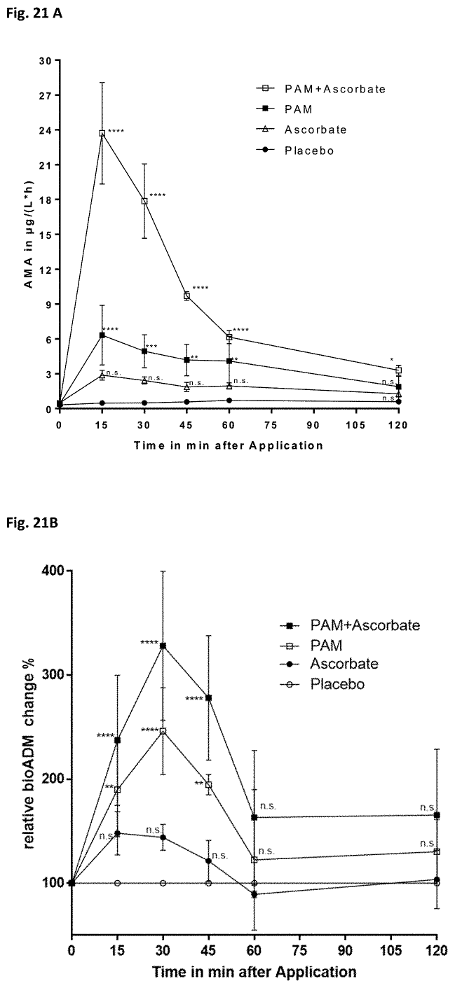

AMA in plasma samples was determined as described in example 3. AMA prior to application was defined as 100% and AMA after application was normalized to AMA prior to application. AMA (%) from the three animals is shown in FIG. 19. Half-Life of the PAM enzyme was determined from the averaged activities of the Enzyme group animals (FIG. 20) using a one phase decay fit (Graph Pad Prism). The half-life of the PAM Enzyme was 47 min.

In a second experiment three animals (Wistar-rat, male, 2-3 month of age) received 25 μg of recombinant human PAM (see example 3) as a single dose in a total volume of 500 μl. One control-animal received 500 μl of PBS. Blood sampling (Li-Heparin Plasma) was carried out 15 minutes prior to application and 15, 30, 45, 60, 90 min as well as 2 h, 3 h, 4 h, and 8 h, respectively, after application. Half-life of the PAM enzyme was determined as described above and was with 60 min comparable to the determination described above.

These data clearly demonstrate the capability of intravenously administered recombinant human PAM to convert circulating ADM-Gly into bio-ADM in vivo. The administration of recombinant human PAM led to a bio-ADM increase with a maximum after 4 h with a half-life of the PAM enzyme of approximately 53.5 min.

### Example 10—Injection of Recombinant PAM in Combination with Ascorbate in Rats

Endotoxin-free, recombinant PAM (SinoBiological) was buffer exchanged into sterile phosphate buffer saline (PBS, Dulbecco) and adjusted to 50 μg/mL. Sterile ascorbate solution for injection (200 mg/mL, vitamin C 1000, WÖRWAG Pharma) was purchased in a local pharmacy and adjusted under sterile conditions with PBS to 40 mg/mL. For combined injections of PAM and ascorbate, the compounds were prepared separately in equal volumes (100 μg/mL or 80 mg/mL for PAM and ascorbate, respectively). Both compounds were combined directly prior to injection to result in concentrations of 50 μg/mL for PAM and 40 mg/mL for ascorbate. Sterile PBS was used as placebo. All samples were stored at −80° C. until use. Animals, male Wistar rats, 2-3 month of age, were assigned into 4 groups (placebo, ascorbate, PAM, PAM+ascorbate) with 3 animals per group. Each animal in the individual group received 500 μl of the respective compound intravenously as a single dose injection. Blood sampling was performed as Li-Heparin 30 min prior to and 15, 30, 45, 60, 120, 180, 240 and 300 min post injection. PAM activities were determined as described in example 3.

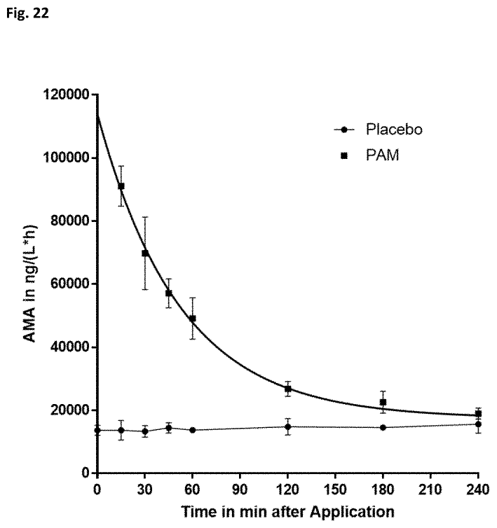

Injection of Ascorbate in rats resulted in non-significant elevation of endogenous PAM activity, when measured without exogenous Ascorbate addition in assay (FIG. 21A, open triangles). PAM Activity remained elevated for 60 minutes after injection with its maximal elevation after 15 min past injection. After 120 min past injection measured activity was comparable to PAM activity in the placebo group. Injection of recombinant human PAM in rats resulted in highly significant elevation of circulating PAM activity, when measured without exogenous Ascorbate addition (FIG. 21A, filled squares). Maximal elevation of PAM activity by the factor of 6 when compared to placebo group was reached 15 minutes past injection. Circulating PAM activity remained significantly elevated for 60 minutes after injection (30 min, 45 min and 60 min) with a decreasing tendency in this time period. After 120 min past injection measured PAM activity was not significantly different to PAM activity in the placebo group. Injection of a combination of recombinant human PAM and Ascorbate in rats resulted in highly significant elevation of circulating PAM activity, when measured without exogenous Ascorbate addition (FIG. 21A, open squares). Maximal elevation of PAM activity by the factor of 24 when compared to placebo group was reached 15 minutes past injection. Circulating PAM activity remained significantly elevated for 60 minutes after injection (30 min, 45 min, 60 min) with a decreasing tendency in this time period. After 120 min past injection measured activity remained significantly elevated when compared to PAM activity in the placebo group. Application of enzyme- and ascorbate-free placebo did not increase the circulating PAM Activity (FIG. 21A, open circles).

The Determination of Bio-ADM in Circulation of all Treatment-Groups Surprisingly Revealed Elevation of Circulating Bio-ADM Concentration in a Time-Dependent Manner:

After application of Ascorbate, bio-ADM concentration was elevated (non-significant) by the factor of 1.4 after 15 min and after 30 min, by the factor of 1.2 after 45 min and returned to baseline levels after 60 minutes (FIG. 21B, filled circles). After application of recombinant human PAM (FIG. 21B, open squares), bio-ADM concentration was highly significantly elevated by the factor of 1.8 after 15 min and by the factor of 2.4 after 30 min. After 45 min bio-ADM concentrations decreased but remained significantly elevated by the factor of 2. After 60 min bio-ADM concentrations were not significantly different when compared to the placebo group. Highest elevation of circulating bio-ADM was observed in the PAM+Ascorbate combination group (FIG. 21B, filled squares): After application of recombinant human PAM and Ascorbate, bio-ADM concentration was highly significantly elevated by the factor of 2.3 after 15 min and by the factor of 3.2 after 30 min. After 45 min bio-ADM concentrations decreased but remained significantly elevated by the factor of 2.8. After 60 min bio-ADM concentrations remained elevated by the factor of 1.6, but the difference was not significant when compared the placebo group. In all 3 groups (Ascorbate, PAM and PAM+Ascorbate) bio-ADM concentrations were not significantly different to the Placebo group after 120 minutes. Application of enzyme- and Ascorbate-free placebo did not increase the bio-ADM concentration in plasma (FIG. 21B, open circles). Bio-ADM concentrations of the placebo group were set as 100% for each timepoint and bio-ADM concentrations in the treatment groups were normalized to the placebo group.

These data clearly demonstrate the capability of intravenously administered recombinant human PAM and a combination of recombinant human PAM with Ascorbate to convert circulating ADM-Gly into bio-ADM in vivo. The administration of recombinant human PAM led to a bio-ADM increase with a maximum after 30 minutes.

As shown in FIG. 22, the half-life of recombinant human PAM in rats is 52.7 minutes. The determined half-life is comparable to the half-life determined in example 9 (FIG. 20), while the precision of the half-life determination was increased by generation of additional datapoints between 0 and 60 minutes after injection

### Example 11—Effect of Ascorbate on Circulating Human PAM Activity

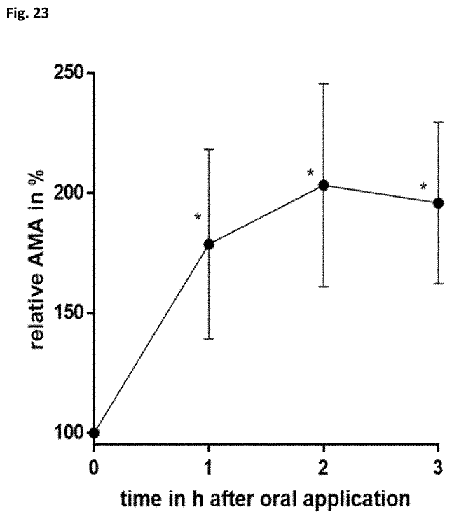

Healthy volunteers (n=4) received 2000 mg of vitamin C (Dr. Scheffler, Additiva® Vitamin C) as an oral single dose. Blood sampling was performed prior to- and 1, 2 and 3 hours post administration. Basal amidating activity was determined from Li-heparin plasma and serum as described in example 3 in absence of exogenous ascorbate addition and is shown in FIG. 23.

Surprisingly, PAM activity determined without exogenous ascorbate addition was significantly elevated 1 h after oral Ascorbate uptake by the factor of 1.7 when compared to PAM activity prior to ascorbate uptake (FIG. 23). Further, after 2 h and 3 h post oral ascorbate uptake, PAM activity remained elevated by the factor of ˜2. These data clearly demonstrate that oral Ascorbate uptake is suitable for modulation of circulating PAM activity in humans.

SEQUENCES

```
SEQ ID NO: 1-Prepro-PAM isoform 1 AS 1-973
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QEKQKLIKEP GSGVPVVLIT TLLVIPVVVL LAIAIFIRWK KSRAFGDSEH
              910 920 930 940 950
KLETSSGRVL GRFRGKGSGG LNLGNFFASR KGYSRKGFDR LSTEGSDQEK
              960 970
EDDGSESEEE YSAPLPALAP SSS
SEQ ID NO: 2-Prepro-PAM isoform 2 AS 1-868
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQDFH MEEALDWPGV
              410 420 430 440 450
YLLPGQVSGV ALDPKNNLVI FHRGDHVWDG NSFDSKFVYQ QIGLGPIEED
              460 470 480 490 500
TILVIDPNNA AVLQSSGKNL FYLPHGLSID KDGNYWVTDV ALHQVFKLDP
              510 520 530 540 550
NNKEGPVLIL GRSMQPGSDQ NHFCQPTDVA VDPGTGAIYV SDGYCNSRIV
              560 570 580 590 600
QFSPSGKFIT QWGEESSGSS PLPGQFTVPH SLALVPLLGQ LCVADRENGR
              610 620 630 640 650
IQCFKTDTKE FVREIKHSSF GRNVFAISYI PGLLFAVNGK PHFGDQEPVQ
              660 670 680 690 700
GFVMNFSNGE IIDIFKPVRK HFDMPHDIVA SEDGTVYIGD AHTNTVWKFT
              710 720 730 740 750
LTEKLEHRSV KKAGIEVQEI KEAEAVVETK MENKPTSSEL QKMQEKQKLI
              760 770 780 790 800
KEPGSGVPVV LITTLLVIPV VVLLAIAIFI RWKKSRAFGD SEHKLETSSG
              810 820 830 840 850
RVLGRFRGKG SGGLNLGNFF ASRKGYSRKG FDRLSTEGSD QEKEDDGSES
              860
EEEYSAPLPA LAPSSS
SEQ ID No.: 3-Prepro-PAM isoform 3 AS (amino acids 829-896
of SEQ ID No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKDS EHKLETSSGR VLGRFRGKGS
              860 870 880 890 900
GGLNLGNFFA SRKGYSRKGF DRLSTEGSDQ EKEDDGSESE EEYSAPLPAL
    905
APSSS
SEQ ID No. 4-Prepro-PAM isoform 4 (amino acids 829-914 of
SEQ ID No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKGK GSGGLNLGNF FASRKGYSRK
              860 870 880
GFDRLSTEGS DQEKEDDGSE SEEEYSAPLP ALAPSSS
SEQ ID No. 5-Prepro-PAM Isoform 5 (Isoform 1 with an
additional aa in position 896)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QEKQKLIKEP GSGVPVVLIT TLLVIPVVVL LAIAIFIRWK KSRAFGADSE
              910 920 930 940 950
HKLETSSGRV LGRFRGKGSG GLNLGNFFAS RKGYSRKGFD RLSTEGSDQE
              960 970
KEDDGSESEE EYSAPLPALA PSSS
SEQ ID No. 6-Prepro-PAM Isoform 6 (amino acids 897-914 of
SEQ ID No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QEKQKLIKEP GSGVPVVLIT TLLVIPVVVL LAIAIFIRWK KSRAFGGKGS
              910 920 930 940 950
GGLNLGNFFA SRKGYSRKGF DRLSTEGSDQ EKEDDGSESE EEYSAPLPAL
APSSS
SEQ ID No. 7-PHM subunit of PAM
                10 20 30 40 50
FKETTRPFSN ECLGTTRPVV PIDSSDFALD IRMPGVTPKQ SDTYFCMSMR
                60 70 80 90 100
IPVDEEAFVI DFKPRASMDT VHHMLLFGCN MPSSTGSYWF CDEGTCTDKA
              110 120 130 140 150
NILYAWARNA PPTRLPKGVG FRVGGETGSK YFVLQVHYGD ISAFRDNNKD
              160 170 180 190 200
CSGVSLHLTR LPQPLIAGMY LMMSVDTVIP AGEKVVNSDI SCHYKNYPMH
              210 220 230 240 250
VFAYRVHTHH LGKVVSGYRV RNGQWTLIGR QSPQLPQAFY PVGHPVDVSF
              260 270 280 290 300
GDLLAARCVF TGEGRTEATH IGGTSSDEMC NLYIMYYMEA KHAVSFMTCT
              310 320 330 340 350
QNVAPDMFRT IPPEANIPIP VKSDMVMMHE HHKETEYKDK IPLLQQPKRE
              360 370 380 390 400
EEEVLDQGDF YSLLSKLLGE REDVVHVHKY NPTEKAESES DLVAEIANVV
              410 420 430 440 450
QKKDLGRSDA REGAEHERGN AILVRDRIHK FHRLVSTLRP PESRVFSLQQ
              460
PPPGEGTWEP EHTG
SEQ ID No. 8-PAL subunit of PAM
                10 20 30 40 50
DFHMEEALDW PGVYLLPGQV SGVALDPKNN LVIFHRGDHV WDGNSFDSKF
                60 70 80 90 100
VYQQIGLGPI EEDTILVIDP NNAAVLQSSG KNLFYLPHGL SIDKDGNYWV
              110 120 130 140 150
TDVALHQVFK LDPNNKEGPV LILGRSMQPG SDQNHFCQPT DVAVDPGTGA
              160 170 180 190 200
IYVSDGYCNS RIVQFSPSGK FITQWGEESS GSSPLPGQFT VPHSLALVPL
              210 220 230 240 250
LGQLCVADRE NGRIQCFKTD TKEFVREIKH SSFGRNVFAI SYIPGLLFAV
              260 270 280 290 300
NGKPHFGDQE PVQGFVMNFS NGEIIDIFKP VRKHFDMPHD IVASEDGTVY
              310 320
IGDAHTNTVW KFTLTEKLEH RSV
SEQ ID No. 9-signal sequence human serum albumin
                10 20
MKWVTFISLL FLFSSAYSFR
SEQ ID No. 10-Sequence of recombinant human PAM
                10 20 30 40 50
SPLSVFKRFK ETTRPFSNEC LGTTRPVVPI DSSDFALDIR MPGVTPKQSD
                60 70 80 90 100
TYFCMSMRIP VDEEAFVIDF KPRASMDTVH HMLLFGCNMP SSTGSYWFCD
              110 120 130 140 150
EGTCTDKANI LYAWARNAPP TRLPKGVGFR VGGETGSKYF VLQVHYGDIS
              160 170 180 190 200
AFRDNNKDCS GVSLHLTRLP QPLIAGMYLM MSVDTVIPAG EKVVNSDISC
              210 220 230 240 250
HYKNYPMHVF AYRVHTHHLG KVVSGYRVRN GQWTLIGRQS PQLPQAFYPV
              260 270 280 290 300
GHPVDVSFGD LLAARCVFTG EGRTEATHIG GTSSDEMCNL YIMYYMEAKH
              310 320 330 340 350
AVSFMTCTQN VAPDMFRTIP PEANIPIPVK SDMVMMHEHH KETEYKDKIP
              360 370 380 390 400
LLQQPKREEE EVLDQGDFYS LLSKLLGERE DVVHVHKYNP TEKAESESDL
              410 420 430 440 450
VAEIANVVQK KDLGRSDARE GAEHERGNAI LVRDRIHKFH RLVSTLRPPE
              460 470 480 490 500
SRVFSLQQPP PGEGTWEPEH TGDFHMEEAL DWPGVYLLPG QVSGVALDPK
              510 520 530 540 550
NNLVIFHRGD HVWDGNSFDS KFVYQQIGLG PIEEDTILVI DPNNAAVLQS
              560 570 580 590 600
SGKNLFYLPH GLSIDKDGNY WVTDVALHQV FKLDPNNKEG PVLILGRSMQ
              610 620 630 640 650
PGSDQNHFCQ PTDVAVDPGT GAIYVSDGYC NSRIVQFSPS GKFITQWGEE
              660 670 680 690 700
SSGSSPLPGQ FTVPHSLALV PLLGQLCVAD RENGRIQCFK TDTKEFVREI
              710 720 730 740 750
KHSSFGRNVF AISYIPGLLF AVNGKPHFGD QEPVQGFVMN FSNGEIIDIF
              760 770 780 790 800
KPVRKHFDMP HDIVASEDGT VYIGDAHTNT VWKFTLTEKL EHRSVKKAGI
              810
EVQEIKEAEA VVGS
SEQ ID No. 11-Peptide 1 (aa 42-56 of PAM SEQ ID No. 1)
                10
CLGTTRPVVP IDSSD
SEQ ID No. 12-Peptide 2 (aa 109-128 of PAM SEQ ID No. 1)
                10
CNMPSSTGSY WFCDEGTCTD
SEQ ID No. 13-Peptide 3 (aa 168-180 of PAM SEQ ID No. 1)
                10
YGDISAFRDN NKD
SEQ ID No. 14-Peptide 4 (aa 204-216 of PAM SEQ ID No. 1)
                10
SVDTVIPAGE KVV
SEQ ID No. 15-Peptide 5 (aa 329-342 of PAM SEQ ID No. 1)
                10
CTQNVAPDMF RTIP
SEQ ID No. 16-Peptide 6 (aa 291-310 of PAM SEQ ID No. 1)
                10 20
TGEGRTEATH IGGTSSDEMC
SEQ ID No. 17-Peptide 7 (aa 234-244 of PAM SEQ ID No. 1)
                10
YRVHTHHLGK V
SEQ ID No. 18-Peptide 8 (aa 261-276 of PAM SEQ ID No. 1)
                10
QSPQLPQAFY PVGHPV
SEQ ID No. 19-Peptide 9 (aa 530-557 of PAM SEQ ID No. 1)
                10 20
RGDHVWDGNS FDSKFVYQQI GLGPIEED
SEQ ID No. 20-Peptide 10 (aa 611-631 of PAM SEQ ID No. 1)
                10 20
EGPVLILGRS MQPGSDQNHF C
SEQ ID No. 21-Peptide 11 (aa 562-579 of PAM SEQ ID No. 1)
                10
IDPNNAAVLQ SSGKNLFY
SEQ ID No. 22-Peptide 12 (aa 745-758 of PAM SEQ ID No. 1)
                10
NGKPHFGDQE PVQG
SEQ ID No. 23-Peptide 13 (aa 669-687 of PAM SEQ ID No. 1)
                10
WGEESSGSSP LPGQFTVPH
SEQ ID No. 24-Peptide 14 (aa 710-725 of PAM SEQ ID No. 1)
                10
CFKTDTKEFV REIKHS
```

1. Peptidylglycine alpha-amidating monooxygenase (PAM) for use as a medicament.

2. PAM for use as a medicament for treatment of a subject, wherein said treatment comprises:
(i) reducing the potential or risk for a disease or disorder, and/or
(ii) reducing the occurrence of a disease or disorder, and/or
(iii) reducing the severity of a disease or disorder.

3. PAM for use as a medicament for treatment of a subject according to claim 2, wherein said disease or disorder is selected from the group comprising dementia, cardiovascular disorders, kidney diseases, cancer, inflammatory or infectious diseases and/or metabolic diseases.

4. PAM for use as a medicament for treatment of a subject according to claim 2, wherein said subject is characterized by
a level of PAM and/or its isoforms and/or fragments thereof below a threshold and/or
a peptide-Gly/peptide-amide ratio above a threshold
in a sample of bodily fluid of said subject.

5. PAM for use as a medicament for treatment of a subject according to claim 4, wherein said peptide is selected from the group of comprising adrenomedullin (ADM), adrenomedullin-2, intermedin-short, pro-adrenomedullin N-20 terminal peptide (PAMP), amylin, gastrin releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PA CAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin, vasopressin.

6. PAM for use as a medicament for treatment of a subject according to claim 4, wherein said subject is characterized by
an ADM-Gly/bio-ADM ratio above a threshold and/or
a bio-ADM concentration below a threshold
in a bodily fluid of said patient.

7. PAM for use as a medicament for treatment of a subject according to claim 3, wherein the level of PAM and/or its isoforms and/or fragments thereof is the total concentration of PAM and/or its isoforms and/or fragments thereof having at least 12 amino acids or the activity of PAM and/or its isoforms and/or fragments thereof.

8. PAM for use as a medicament for treatment of a subject according to claim 7, wherein the total concentration of PAM and/or its isoforms and/or fragments thereof having at least 12 amino acids or the activity of PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

9. PAM for use as a medicament for treatment of a subject according to claim 4, wherein the sample of bodily fluid of said subject is selected from the group of blood, serum, plasma, urine, cerebrospinal fluid (CSF), and saliva.

10. PAM for use as a medicament according to claim 1, wherein said PAM is selected from the group comprising isolated and/or recombinant and/or chimeric PAM.

11. PAM for use as a medicament according to claim 1, wherein said recombinant PAM is selected from the sequences comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

12. PAM for use as a medicament for treatment of a subject according to any embodiment 1 to 11 wherein PAM is combined with ascorbate and/or copper.

13. Pharmaceutical formulation comprising peptidylglycine alpha-amidating monooxygenase (PAM).

14. Pharmaceutical formulation comprising PAM according to claim 13, wherein said pharmaceutical formulation is administered orally, epicutaneously, subcutaneously, intradermally, sublingually, intramuscularly, intraarterially, intravenously, or via the central nervous system (CNS, intracerebrally, intracerebroventricularly, intrathecally) or via intraperitoneal administration.

15. Pharmaceutical formulation according to claim 13, wherein said pharmaceutical formulation is a solution, preferably a ready-to-use solution.

16. Pharmaceutical formulation according to claim 13, wherein said pharmaceutical formulation is in a freeze-dried state.

17. Pharmaceutical formulation according to claim 13, wherein said pharmaceutical formulation is administered intra-muscular.

18. Pharmaceutical formulation according to claim 13, wherein said pharmaceutical formulation is administered intra-vascular.

19. Pharmaceutical formulation according to claim 13, wherein said pharmaceutical formulation is administered via infusion.

20. Pharmaceutical formulation according to claim 13, wherein said pharmaceutical formulation is to be administered systemically.

21. Pharmaceutical formulation according to claim 13, the formulation comprising PAM and/or optionally one or more pharmaceutically acceptable ingredients

22. Pharmaceutical formulation according to claim 13, the formulation comprising PAM, ascorbate and/or copper.

23. Pharmaceutical formulation according to claim 13, the formulation comprising PAM in combination with ascorbate and/or copper.


SEQUENCE LISTING (24 sequences)

SEQ ID NO: 1 - protein, 973 aa, Homo sapiens [sequence omitted]

SEQ ID NO: 2 - protein, 866 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGDSEHKLETSSGRVLGRFRGKGSGGLNLGNFFASRKGYSRKGFDRLSTEGSDQEKEDDGSESEEEYSAPLPALAPSSS

SEQ ID NO: 3 - protein, 905 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKDSEHKLETSSGRVLGRFRGKGSGGLNLGNFFASRKGYSRKGFDRLSTEGSDQEKEDDGSESEEEYSAPLPALAPSSS

SEQ ID NO: 4 - protein, 887 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKGKGSGGLNLGNFFASRKGYSRKGFDRLSTEGSDQEKEDDGSESEEEYSAPLPALAPSSS

SEQ ID NO: 5 - protein, 974 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGADSEHKLETSSGRVLGRFRGKGSGGLNLGNFFASRKGYSRKGFDRLSTEGSDQEKEDDGSESEEEYSAPLPALAPSSS

SEQ ID NO: 6 - protein, 955 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGGKGSGGLNLGNFFASRKGYSRKGFDRLSTEGSDQEKEDDGSESEEEYSAPLPALAPSSS

SEQ ID NO: 7 - protein, 464 aa, Homo sapiens
FKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTG

SEQ ID NO: 8 - protein, 323 aa, Homo sapiens
DFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSV

SEQ ID NO: 9 - protein, 20 aa, Homo sapiens
MKWVTFISLLFLFSSAYSFR

SEQ ID NO: 10 - protein, 814 aa, Homo sapiens
SPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVGS

SEQ ID NO: 11 - protein, 15 aa, Artificial Sequence
  Peptide
CLGTTRPVVPIDSSD

SEQ ID NO: 12 - protein, 20 aa, Artificial Sequence
  Peptide
CNMPSSTGSYWFCDEGTCTD

SEQ ID NO: 13 - protein, 13 aa, Artificial Sequence
  Peptide
YGDISAFRDNNKD

SEQ ID NO: 14 - protein, 13 aa, Artificial Sequence
  Peptide
SVDTVIPAGEKVV

SEQ ID NO: 15 - protein, 14 aa, Artificial Sequence
  Peptide
CTQNVAPDMFRTIP

SEQ ID NO: 16 - protein, 20 aa, Artificial Sequence
  Peptide
TGEGRTEATHIGGTSSDEMC

SEQ ID NO: 17 - protein, 11 aa, Artificial Sequence
  Peptide
YRVHTHHLGKV

SEQ ID NO: 18 - protein, 16 aa, Artificial Sequence
  Peptide
QSPQLPQAFYPVGHPV

SEQ ID NO: 19 - protein, 28 aa, Artificial Sequence
  Peptide
RGDHVWDGNSFDSKFVYQQIGLGPIEED

SEQ ID NO: 20 - protein, 21 aa, Artificial Sequence
  Peptide
EGPVLILGRSMQPGSDQNHFC

SEQ ID NO: 21 - protein, 18 aa, Artificial Sequence
  Peptide
IDPNNAAVLQSSGKNLFY

SEQ ID NO: 22 - protein, 14 aa, Artificial Sequence
  Peptide
NGKPHFGDQEPVQG

SEQ ID NO: 23 - protein, 19 aa, Artificial Sequence
  Peptide
WGEESSGSSPLPGQFTVPH

SEQ ID NO: 24 - protein, 16 aa, Artificial Sequence
  Peptide
CFKTDTKEFVREIKHS